(19) United States
(12) Patent Application Publication
(10) Pub. No.: US 2024/0249847 A1
(43) Pub. Date: Jul. 25, 2024

(54) OMICS-INFERRED BODY INDEX METHOD AND SYSTEM

(71) Applicant: Institute for Systems Biology, Seattle, WA (US)

(72) Inventors: Noa Rappaport Kengo Watanabe, Seattle, WA (US); Tomasz Wilmanski, Seattle, WA (US); Nathan Price, Seattle, WA (US)

(73) Assignee: Institute for Systems Biology, Seattle, WA (US)

(21) Appl. No.: 18/511,862

(22) Filed: Nov. 16, 2023

Related U.S. Application Data
(60) Provisional application No. 63/480,814, filed on Jan. 20, 2023.

(51) Int. Cl. G16H 50/30

(52) U.S. Cl. CPC G16H 50/30

(57) ABSTRACT

Provided are computer-implemented methods, systems and products of determining omic body index and class of a subject.

### CLAIM FOR PRIORITY

This application claims priority to U.S. Provisional Patent Application No. 63/480,814, filed Jan. 20, 2023, titled “OMICS-INFERRED BODY INDEX METHOD AND SYSTEM,” and which is incorporated by reference in its entirety for all purposes.

### GOVERNMENT LICENSE RIGHTS

This invention was made with government support under U19AG023122 and 5U01AG061359 awarded by the National Institute on Aging (NIA) of the National Institutes of Health (NIH). The government has certain rights in the invention.

### FIELD

This disclosure relates to determining an omics-inferred body index and class of a subject.

### BACKGROUND

Obesity has been increasing in prevalence over the past four decades in adults, adolescents, and children around most of the world. Many studies have demonstrated that obesity is a major risk factor for multiple chronic diseases such as type 2 diabetes mellitus (T2DM), metabolic syndrome (MetS), cardiovascular disease (CVD), and certain types of cancer. In individuals with obesity, even a 5% loss in body weight can improve metabolic and cardiovascular health, and weight loss through lifestyle interventions (e.g., dietary intervention, exercise) can reduce the risk for obesity-related chronic diseases. Nevertheless, obesity and its physiological manifestations can vary widely across individuals, necessitating additional researches to better understand this prevalent health condition.

Obesity is commonly quantified using the anthropometric Body Mass Index (BMI), defined as the body weight divided by body height squared [kg m-2]. While BMI does not directly measure body composition, BMI correlates well at the population level with the body fat percentage measured by specialized devices such as dual-energy X-ray absorptiometry (DXA). As an easily calculated and commonly understood measure among researchers, clinicians, and the general public, BMI is widely used for the primary diagnosis of obesity, and changes in BMI are often used to assess the effectiveness of lifestyle interventions.

There are limitations to BMI as a surrogate measure of health state. Differences in body composition can lead to misclassification of people with a high muscle-to-fat ratio (e.g., athletes) as an individual with obesity, and can undervalue metabolic improvements in health following exercise. A meta-analysis showed that the common obesity diagnoses based on BMI cutoffs had high specificity but low sensitivity in identifying individuals with excess body fat. The misclassification is likely due, in part, to the differences in BMI thresholds for obesity across different ethnic populations, as well as the existence of a metabolically unhealthy, normal-weight (MUNW) group within the normal BMI class. Likewise, there are health-heterogeneous groups among the individuals with obesity: metabolically healthy obese (MHO) and metabolically unhealthy obese (MUO). While most individuals in the MHO group are not necessarily healthy but simply healthier than individuals in the MUO group, the transition from MHO to MUO phenotype may be a preceding step to the development of obesity-related chronic diseases. Moreover, this transition is potentially preventable through lifestyle interventions. Hence, BMI is unequivocally useful at the population level, but too crude to capture a variety of heterogeneous metabolic health states.

Omics studies have demonstrated how blood omic profiles contain information relevant to a wide range of human health conditions; e.g., blood proteomics captured 11 health indicators such as the liver fat measured by ultrasound and the body composition measured by DXA, while blood metabolomics tended to reflect dietary intake, lifestyle patterns, and gut microbiome profiles. A machine learning model that was trained to predict BMI using 49 BMI-associated blood metabolites captured obesity-related clinical measurements (e.g., insulin resistance, visceral fat percentage) better than observed BMI or genetic predisposition for high BMI. Moreover, another blood metabolomics-based model of BMI reflected differences between individuals with or without acute coronary syndrome. Thus, while a single targeted metric (e.g., body composition) or a specific biomarker (e.g., leptin, adiponectin provides useful information, multiomic blood profiling has the potential to comprehensively bridge the multifaceted gaps between BMI and heterogeneous physiological states.

### SUMMARY

Computer-implemented methods for determining an omics-inferred body mass index are provided. The computer-implemented method includes one or more processors programmed to perform a series of steps, comprising:
(a) accessing blood analyte omics data of the subject;
(b) generating an omics body index for the subject by applying a machine learning model to the subject omics data, the machine learning model fitted to blood analyte omic and anthropomorphic body index data of a reference population, the reference population comprising a heterogeneous mixture of individuals classified by different anthropomorphic body index classes;
(c) classifying the subject by the omics body index class according to the anthropomorphic body index class boundaries; and
(d) outputting the omics body index class for the subject.

Also provided are computer-implemented systems and computer-program products for carrying out aspects of the disclosure.

The present computer-implemented methods, systems and products of the disclosure include several advantages. One is that they can identify heterogeneous metabolic health states which are not captured by other approaches. Another is that the omics-inferred body index classifications are more reflective of actual metabolic health. Yet another is the ability to determine an earlier response to changes in metabolic health. Other advantages include, but are not limited to, the ability to determine durable, lasting effects in metabolic health not revealed by other approaches.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1 shows multivariate, omics-inferred metabolic BMI captured 48-78% of the variance in standard BMI.

FIG. 2 shows multivariate, omics-inferred metabolic BMI estimates captured the variance in standard BMI better than any single analyte.

FIG. 3 shows metabolic heterogeneity was responsible for the high rate of misclassification within the standard BMI classes.

FIG. 4 shows metabolomics-inferred BMI reflected gut microbiome profiles better than BMI.

FIG. 5 shows metabolic health of the metabolically obese group was substantially improved following a healthy lifestyle intervention.

FIG. 6 shows plasma analyte correlation network in the metabolically obese group shifted toward a structure observed in metabolically healthier state following a healthy lifestyle intervention.

FIG. 7 shows demographic information of study cohorts.

FIG. 8 shows quality check of the LASSO modeling.

FIG. 9 shows restricted metabolomics-based BMI model predominantly maintained characteristics of the original full model.

FIG. 10 shows omics-based BMI models were similar between LASSO and other methods.

FIG. 11 shows variable diversity and contribution to the omics-based BMI model were different between omics categories.

FIG. 12 shows the metabolic heterogeneity within the standard BMI classes was validated with TwinsUK cohort.

FIG. 13 shows omics-based WHtR models consistently supported the findings of omics-based BMI models.

FIG. 14 shows predominant commonality with minor specificity was observed between the omics-based BMI and WHtR models.

### DESCRIPTION OF SPECIFIC EMBODIMENTS

Provided are computer-implemented methods and systems for determining an omics-inferred body index of a subject from blood analyte data of the subject. The method comprises: (a) accessing blood analyte data of the subject; (b) generating an omics body index for the subject by applying a machine learning model fitted to blood analyte omic and anthropomorphic body index data of a reference population classified by different anthropomorphic body index classes; (c) classifying the subject by the omics body index class according to the anthropomorphic body index class boundaries; and (d) outputting the omics body index class for the subject. The systems comprise, for example, an analysis pipeline for carrying out the method in a suitable computational environment, such as in the cloud. The computer-program products comprise tangibly embodied in a non-transitory machine-readable storage medium, including instructions configured to cause one or more data processors to perform a set of actions of the disclosure.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing the subject invention, certain terms used in accordance with the invention are described first in greater detail, followed by a description of methods, systems and products, followed by examples of the disclosure.

### I. Terms

Definitions of common terms in computational and data science may be found in: Ranganathan et al. (2018) Encyclopedia of Bioinformatics and Computational Biology: ABC of Bioinformatics, Elsevier (which is hereby incorporated by reference in its entirety for all purposes); Saltz et al. (2017) An introduction to data science, Sage Publications (which is hereby incorporated by reference in its entirety for all purposes); James et al. (2013) An introduction to statistical learning, (Vol. 112, p. 18) New York, Springer (which is hereby incorporated by reference in its entirety for all purposes); and other similar references.

### II. Computer-Implemented Methods and Systems of Determining Omics-Inferred Body Index

As summarized above, provided are computer-implemented methods, systems and products for determining an omics body index classified by an anthropomorphic body index based on blood analyte data alone or in combination with other data.

Some embodiments of the present invention include: a computer-implemented method of determining an omics-inferred anthropomorphic body index of a subject, the computer comprising one or more processors programmed to perform a series of steps, comprising: (a) accessing blood analyte omics data of the subject; (b) generating an omics body index for the subject by applying a machine learning model to the subject omics data, the machine learning model fitted to blood analyte omic and anthropomorphic body index data of a reference population, the reference population comprising a heterogeneous mixture of individuals classified by different anthropomorphic body index classes; (c) classifying the subject by the omics body index class according to the anthropomorphic body index class boundaries; and (d) outputting the omics body index class for the subject.

The anthropomorphic body index may be selected from body mass index (BMI, kg m-2), waist circumference (cm), and waist-to-height ratio (WHtR, unitless).

The anthropomorphic BMI may be a World Health Organization (WHO) standard having class boundaries selected from: underweight <18.5 kg m-2; normal 18.5 to 25 kg m-2; overweight 25 to 30 kg m-2; and obese ≥30 kg m-2.

The WHO anthropomorphic BMI standard may further include class boundaries selected from: severely underweight <16.5 kg/m\hat{ }2; class 1 obesity 30 to <35 kg m-2; class 2 obesity 35 to <40 kg m-2; and class 3 obesity 40 kg m-2 or higher.

The anthropomorphic BMI may be an Asian-Pacific standard having class boundaries selected from: underweight <18.5 kg m-2; normal 18.5 to 22.9 kg m-2; overweight 23 to 24.9 kg m-2; and obese ≥25 kg m-2.

The WHtR may be a United Kingdom National Institute for Health and Care Excellence (NICE) standard having class boundaries selected from: 0.4 to 0.49 WHtR for healthy central adiposity; 0.5 to 0.59 WHtR for increased central adiposity; and, 0.6 or more WHtR for high central adiposity.

The method may further includer outputting feedback on the omics body index class selected from, or comprising: (i) health intervention potential, (ii) recommended health intervention, and (iii) feedback on efficacy of the health intervention potential and/or the recommended health intervention.

The health intervention potential may be weight loss potential and/or omic body index reduction potential, the recommended health intervention may be a lifestyle intervention, and/or the feedback on efficacy may comprise a comparison of the subject omics body index before, after, or before and after the health intervention.

The feedback may be a longitudinal trajectory.

The recommended health intervention may be a lifestyle change, such as regular exercise, prebiotics, probiotics, supplements, and prescribed medical treatment compliance.

The blood analyte omics data of the reference population may comprise a panel of ten or more analytes selected from, or comprising, metabolomic data, proteomic data, or a combination thereof.

Step (a) may further comprise accessing clinical labs data of the subject, and wherein step (b) may further comprise generating an omic body index for the subject by applying the machine learning model to the omics and clinical labs data of the subject, the machine learning model fitted to the blood analyte omic and clinical labs data of the reference population.

The machine learning model may be fitted to omics data comprising, or selected from, metabolomic data (MetBMI model, or MetWHtR in case of WHtR), and proteomic data (ProBMI model), clinical labs data (ChemBMI model), or a combination thereof (CombiBMI model).

The blood analyte omics data of the subject may comprise the metabolomic data and/or analytes co-linear therewith.

The blood analyte omic data of the reference population or the subject may comprise actual and imputed data, such as imputation by random forest regression or k-nearest neighbors (kNN).

### EXAMPLES

The following examples are provided to illustrate certain particular features and/or embodiments. These examples should not be construed to limit the disclosure to the particular features or embodiments described.

### Example 1: Detailed Description of Figures

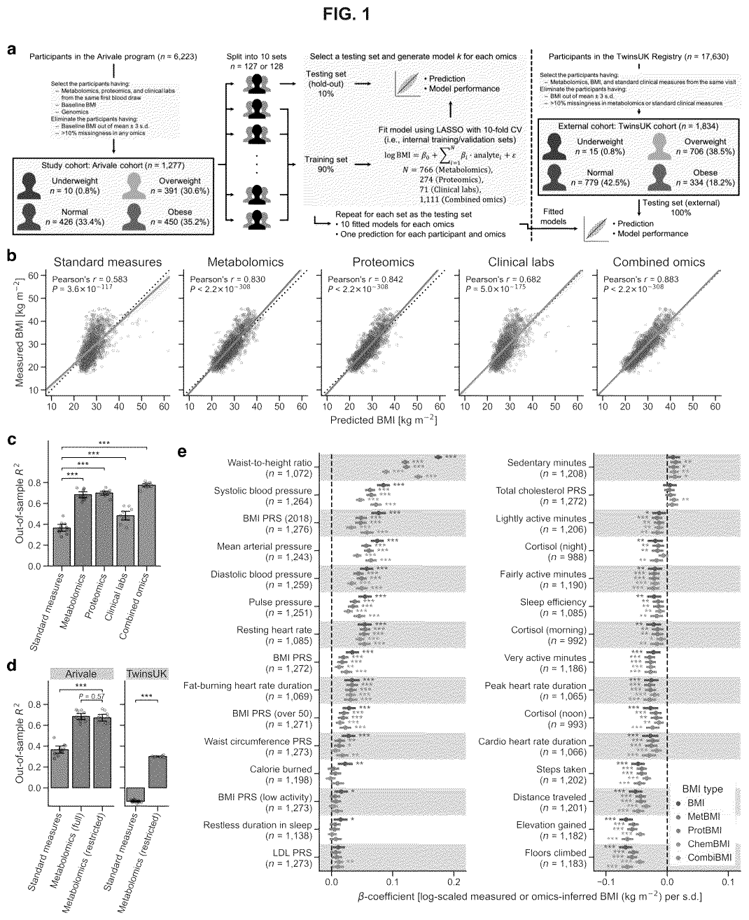

### FIG. 1

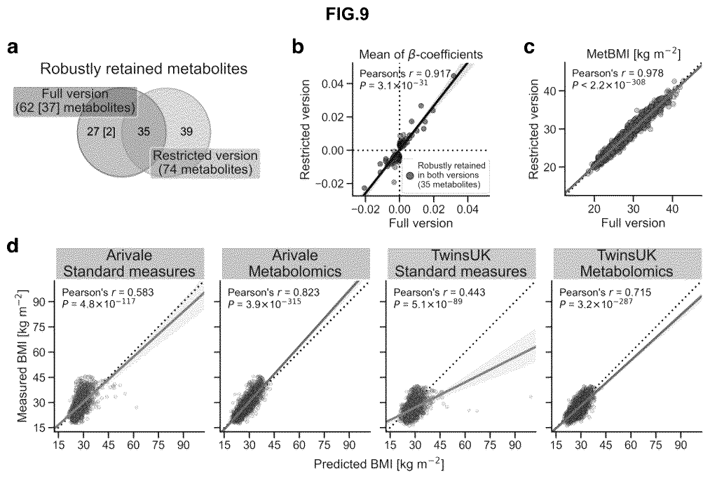

FIG. 1a shows an overview of study cohorts and the omics-based Body Mass Index (BMI) model generation. LASSO: least absolute shrinkage and selection operator, CV: cross-validation. FIG. 1b shows correlation between the measured and predicted BMIs. The solid line is the ordinary least squares (OLS) linear regression line with 95% confidence interval (CI), and the dotted line is measured BMI=predicted BMI. Standard measures: OLS linear regression model with sex, age, triglycerides, high-density lipoprotein (HDL)-cholesterol, low-density lipoprotein (LDL)-cholesterol, glucose, insulin, and homeostatic model assessment for insulin resistance (HOMA-IR) as regressors; P: adjusted P-value of two-sided Pearson's correlation test with the Benjamini-Hochberg method across the five categories. n=1,277 participants. FIG. 1c and FIG. 1d show model performance of each fitted BMI model. Out-of-sample R2 was calculated from each corresponding hold-out testing set (FIG. 1c, Arivale in FIG. 1d) or from the external testing set (TwinsUK in FIG. 1d). Metabolomics (full): LASSO model trained by all 766 metabolites of the Arivale dataset, Metabolomics (restricted): LASSO model trained by the common 489 metabolites in the Arivale and TwinsUK datasets (see FIG. 9). Note that Standard measures and Metabolomics (full) of Arivale in d are the same with corresponding ones in c. Data: mean with 95% CI, n=10 models.

In FIG. 1e, triple asterisks (“***”) indicates adjusted P<0.001 in two-sided Welch's t-test with the Benjamini-Hochberg method across the four (FIG. 1c) or three (FIG. 1d) comparisons. FIG. 1e also shows association between omics-inferred BMI and physiological feature. For each of the 51 numeric physiological features (Supplementary Data 4), β-coefficient was estimated using OLS linear regression model with the measured or omics-inferred BMI as dependent variable and sex, age, and ancestry principal components (PCs) as covariates. Presented are the 30 features that were significantly associated with at least one of the BMI types after multiple testing adjustment with the Benjamini-Hochberg method across the 255 (51 features×5 BMI types) regressions. FIG. 1 key is as follows: (BMI: measured BMI, MetBMI: metabolomics-inferred BMI, ProtBMI: proteomics inferred BMI, ChemBMI: clinical chemistries-inferred BMI, CombiBMI: combined omics-inferred BMI, PRS: polygenic risk score, n: the number of assessed participants); (data: estimate with 95% CI); and (*adjusted P<0.05, **adjusted P<0.01, ***adjusted P<0.001).

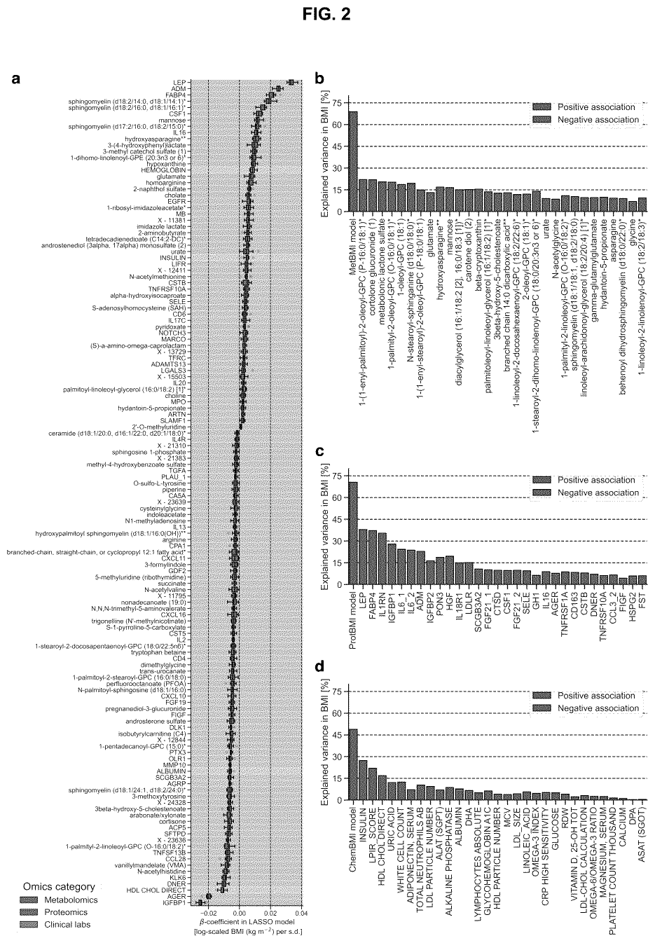

### FIG. 2

FIG. 2a shows the variables that were retained across all ten combined omics-based Body Mass Index (CombiBMI) models (132 analytes: 77 metabolites, 51 proteins, and 4 clinical laboratory tests). β-coefficient was obtained from the fitted CombiBMI model with least absolute shrinkage and selection operator (LASSO) regression. Each background color corresponds to the analyte category. Data: median (center line), [Q1, Q3] (box limits), [xmin, xmax] (whiskers), where Q1 and Q3 are the 1st and 3rd quartile values and xmin, and xmax are the minimum and maximum values in [Q1−1.5×IQR, Q3+1.5×IQR] (IQR: the interquartile range, Q3−Q1), respectively; n=10 models. FIG. 2b through FIG. 2d shows the univariate explained variance in BMI by each metabolite (FIG. 2b), protein (FIG. 2c), or clinical laboratory test (FIG. 2d). BMI was independently regressed on each of the analytes that were retained in at least one of the ten LASSO models (209 metabolites, 74 proteins, 41 clinical laboratory tests; Supplementary Data 5), using ordinary least squares (OLS) linear regression with sex, age, and ancestry principal components (PCs) as covariates. Multiple testing was adjusted with the Benjamini-Hochberg method across the 210 (FIG. 2b), 75 (FIG. 2c), or 42 (FIG. 2d) regressions, including each omics-based BMI (MetBMI: metabolomics-based BMI, ProtBMI: proteomics-based BMI, ChemBMI: clinical chemistries-based BMI) model as reference. Among the analytes that were significantly associated with BMI (180 metabolites, 63 proteins, 30 clinical laboratory tests), only the top 30 significant analytes are presented with their univariate variances.

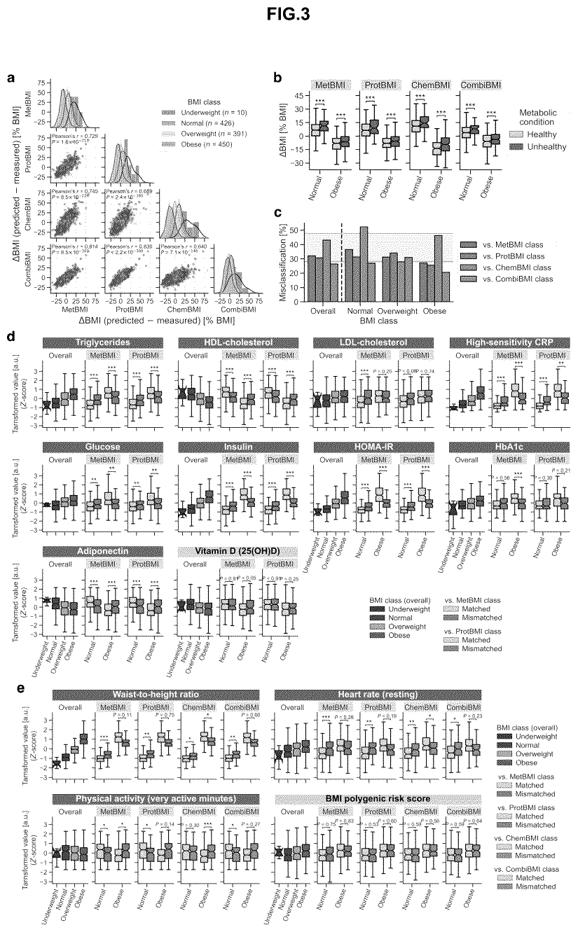

### FIG. 3

FIG. 3a shows the difference of the omics-inferred Body Mass Index (BMI) from the measured BMI (ABMI). MetBMI: metabolomics-inferred BMI, ProtBMI: proteomics-inferred BMI, ChemBMI: clinical chemistries-inferred BMI, CombiBMI: combined omics-inferred BMI, P: adjusted P-value of two sided Pearson's correlation test with the Benjamini-Hochberg method across the six combinations, n=the number of participants in each BMI class (total n=1,277 participants). The line in histogram panels indicates the kernel density estimate. FIG. 3b shows the difference in ABMI between clinically-defined metabolic health conditions within the normal or obese BMI class. Significance was assessed using ordinary least squares (OLS) linear regression with BMI, sex, age, and ancestry principal components (PCs) as covariates, while adjusting multiple testing with the Benjamini-Hochberg method across the eight (two BMI classes×four omics categories) regressions. FIG. 3c shows the misclassification rate of overall cohort or each BMI class against the omics-inferred BMI class. Range of the previously reported misclassification rate is highlighted with orange background. Note that the underweight BMI class is not presented due to small sample size, but its misclassification rate was 80% against CombiBMI class and 100% against the others. FIG. 3d and FIG. 3e show the difference in the obesity-related clinical blood marker (d) or BMI-associated physiological feature (c) between Matched and Mismatched groups within the normal or obese BMI class. Significance was assessed using OLS linear regression with BMI, sex, age, and ancestry PCs as covariates, while adjusting multiple testing with the Benjamini-Hochberg method across the 40 (FIG. 3d, 2 BMI classes×2 omics categories×10 markers) or 216 (FIG. 3c, 2 BMI classes×4 omics categories×27 features) regressions. Four of the 27 features that were significantly associated with BMI (FIG. 1c) are representatively presented in e, and the other results are found in Supplementary Data 6. HDL: high-density lipoprotein, LDL: low-density lipoprotein, CRP: C-reactive protein, HOMA-IR: homeostatic model assessment for insulin resistance, HbA1c: glycated hemoglobin A1c, 25(OH)D: 25-hydroxyvitamin D. FIG. 3b, FIG. 3d, and FIG. 3c data key: median (center line), 95% confidence interval (CI) around median (notch), [Q1, Q3] (box limits), [xmin, xmax] (whiskers), where Q1 and Q3 are the 1st and 3rd quartile values and xmin, and xmax are the minimum and maximum values in [Q1−1.5×IQR, Q3+1.5×IQR] (IQR: the interquartile range, Q3−Q1), respectively; n=373 (FIG. 3b, Healthy in Normal), 49 (FIG. 3b, Unhealthy in Normal), 208 (FIG. 3b, Healthy in Obese), 241 (FIG. 3b, Unhealthy in Obese) participants (see Supplementary Data 6 for each sample size in FIG. 3d and FIG. 3c). *Adjusted P<0.05, **adjusted P<0.01, ***adjusted P<0.001.

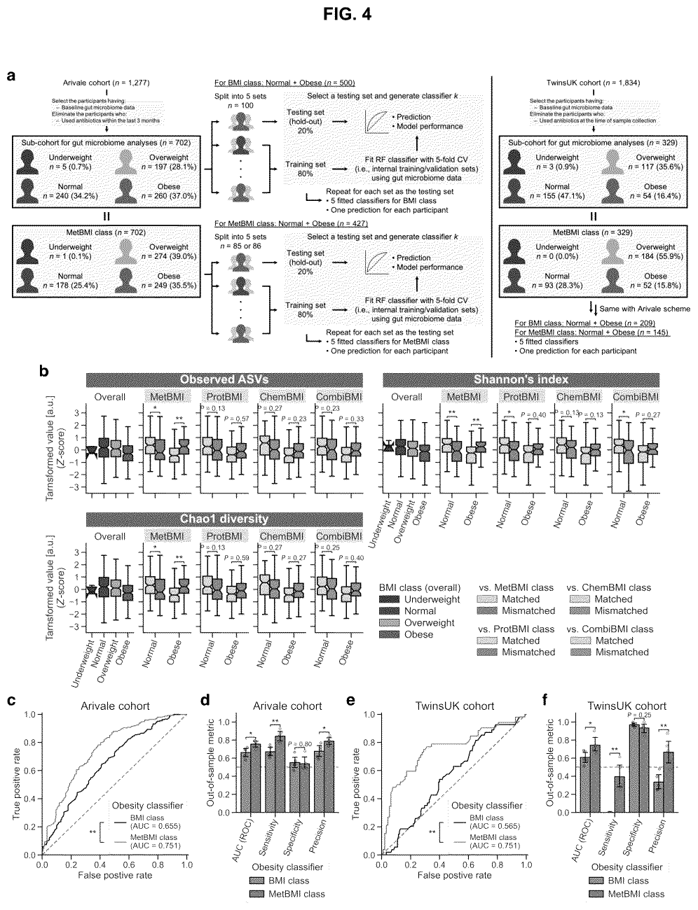

### FIG. 4

FIG. 4a shows an overview of study cohorts and the gut microbiome-based obesity classifier generation. BMI: Body Mass Index, MetBMI: metabolomics-inferred BMI, RF: random forest, CV: cross-validation. FIG. 4b shows the difference in gut microbiome α-diversity between Matched and Mismatched groups within the normal or obese BMI class. Significance was assessed using ordinary least squares (OLS) linear regression with BMI, sex, age, and ancestry principal components (PCs) as covariates, while adjusting multiple testing with the Benjamini-Hochberg method across the 24 (2 BMI classes×4 omics categories×3 metrics) regressions. ProtBMI: proteomics-inferred BMI, ChemBMI: clinical chemistries-inferred BMI, CombiBMI: combined omics-inferred BMI, ASV: amplicon sequence variant. Data: median (center line), 95% confidence interval (CI) around median (notch), [Q1, Q3] (box limits), [xmin, xmax] (whiskers), where Q1 and Q3 are the 1st and 3rd quartile values and xmin, and xmax are the minimum and maximum values in [Q1−1.5×IQR, Q3+1.5×IQR] (IQR: the interquartile range, Q3−Q1), respectively. n=240 (Normal), 260 (Obese) participants (see Supplementary Data 6 for each sample size). *Adjusted P<0.05, **adjusted P<0.01. FIG. 4c and FIG. 4e show receiver operator characteristic (ROC) curve of the gut microbiome-based model classifying participants to the normal vs. obese class in the Arivale (c) or TwinsUK (e) cohort. Each ROC curve was generated from the overall participants: n=500 (FIG. 4c, BMI class), 427 (FIG. 4c, MetBMI class), 209 (FIG. 4c, BMI class), 145 (FIG. 4c, MetBMI class) participants. The red dashed line indicates a random classification line. AUC: area under curve. **P<0.01 in two-sided unpaired DeLong's test. FIG. 4d and FIG. 4f show a comparison of model performance between the BMI and MetBMI classifiers in the Arivale (d) or TwinsUK (f) cohort. Out-of-sample metric value was calculated from each corresponding hold-out testing set. Data: mean with 95% CI, n=5 models. *P<0.05, **P<0.01 in two-sided Welch's t-test.

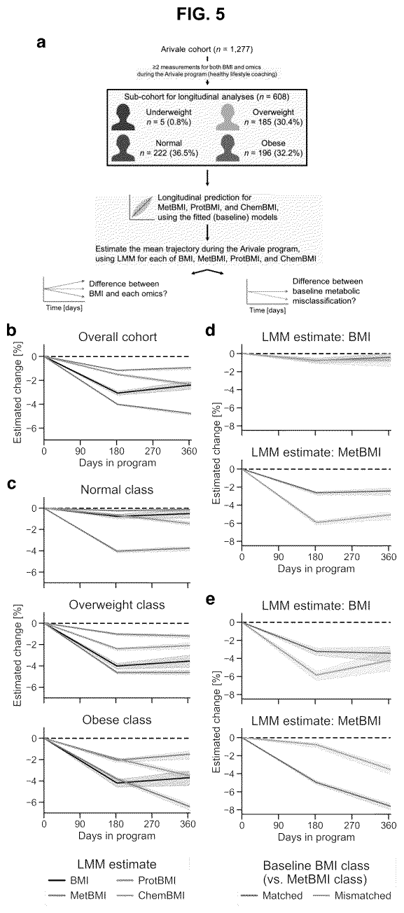

### FIG. 5

FIG. 5a shows an overview of the longitudinal analysis using omics-inferred Body Mass Index (BMI). BMI: measured BMI, MetBMI: metabolomics-inferred BMI, ProtBMI: proteomics-inferred BMI, ChemBMI: clinical chemistries-inferred BMI, LMM: linear mixed model. FIG. 5b and FIG. 5c show longitudinal change in the omics-inferred BMI within the overall cohort (FIG. 5b) or within each baseline BMI class (FIG. 5c). Average trajectory of each measured or omics-inferred BMI was independently estimated using LMM with random effects for each participant (see above methods) in the overall cohort (b) or in each baseline BMI class-stratified group (FIG. 5c). n=608 (FIG. 5b), 222 (FIG. 5c, Normal), 185 (FIG. 5c, Overweight), 196 (FIG. 5c, Obese) participants. FIG. 5d and FIG. 5e show longitudinal change in MetBMI of the misclassified participants within the normal (FIG. 5d) or obese (FIG. 5c) BMI class. Average trajectory of each BMI or MetBMI was independently estimated using the above LMM with the baseline misclassification of BMI class against MetBMI class as additional fixed effects (see above methods) in each baseline BMI class-stratified group. n=137 (FIG. 5d, Matched), 85 (FIG. 5d, Mismatched), 139 (FIG. 5c, Matched), 57 (FIG. 5c, Mismatched) participants. FIG. 5b through FIG. 5e show that the dashed line corresponds to the baseline value of each estimate. Data: mean with 95% confidence interval (CI).

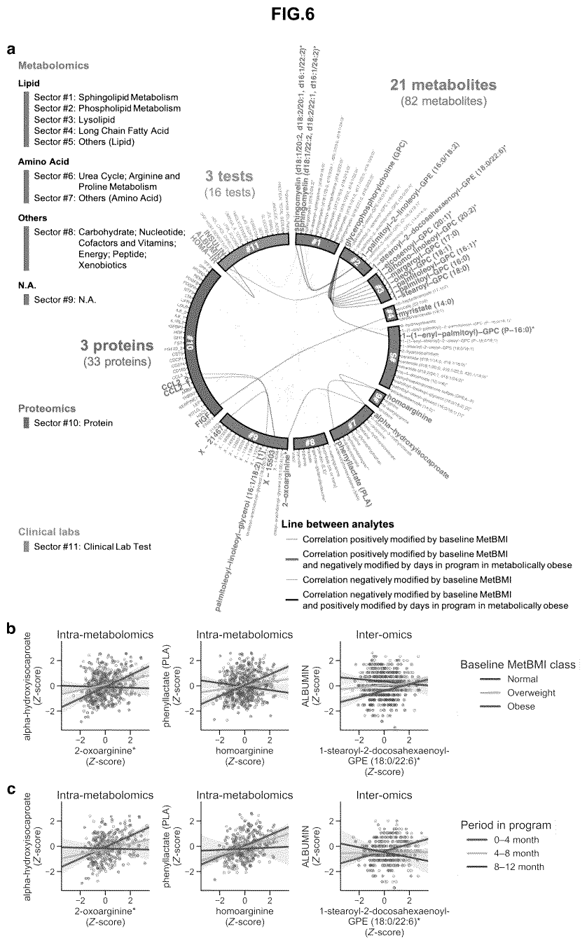

### FIG. 6

FIG. 6a shows cross-omic interactions modified by metabolomics-inferred Body Mass Index (MetBMI) and days in the program. Among 608,856 pairwise relationships of plasma analytes (766 metabolites, 274 proteomics, 64 clinical laboratory tests), 100 analyte-analyte pairs (82 metabolites, 33 proteins, 16 clinical laboratory tests; Supplementary Data 7) that were significantly modified by the baseline MetBMI within the Arivale sub-cohort (FIG. 5a; 608 participants) are presented, whose significance was assessed using their interaction term in each generalized linear model (GLM; see above methods) while adjusting multiple testing with the Benjamini-Hochberg method. Among these significant 100 pairs, 27 analyte-analyte pairs (21 metabolites, 3 proteins, 3 clinical laboratory tests) that were significantly modified by days in the program within the metabolically obese group (i.e., the baseline obese MetBMI class; 182 participants) are highlighted by line width and label font size, whose significance was assessed using their interaction term in each generalized estimating equation (GEE; see above methods) while adjusting multiple testing with the Benjamini-Hochberg method. FIG. 6b and FIG. 6c show representative examples of the analyte-analyte pair that was significantly modified by the baseline MetBMI (FIG. 6b) and days in the program (FIG. 6c) in FIG. 6a. The solid line in each panel is the ordinary least squares (OLS) linear regression line with 95% confidence interval (CI). n=530 (FIG. 6b, Intra-metabolomics (left)), 553 (FIG. 6b, Intra-metabolomics (right)), 566 (FIG. 6b, Inter-omics) participants; n=324 (FIG. 6c, Intra-metabolomics (left)), 339 (FIG. 6c, Intra-metabolomics (right)), 347 (FIG. 6c, Inter-omics) measurements from the 182 participants of the metabolically obese group. Of note, data points outside of plot range are trimmed in these illustrations.

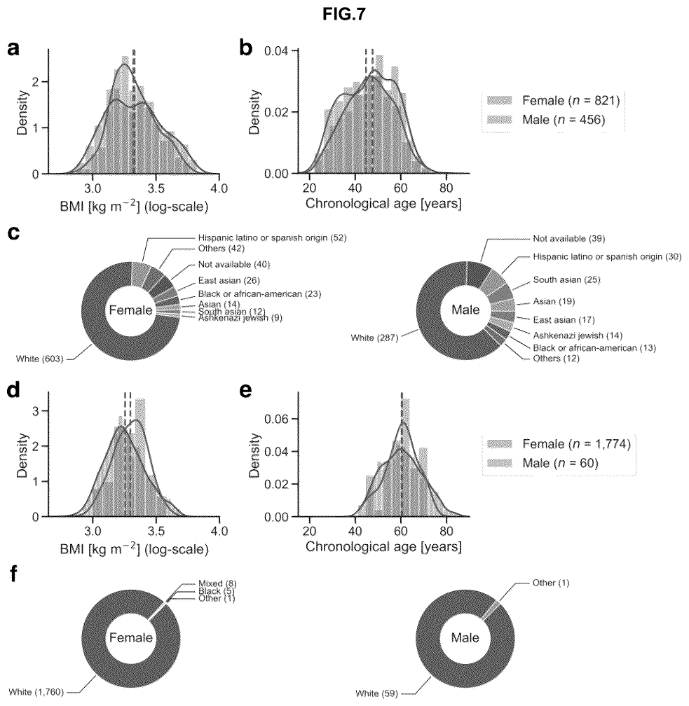

### FIG. 7

FIG. 7 shows demographic information of study cohorts. FIG. 7a-c Demographic information of the Arivale study cohort (FIG. 1a, n=1,277 participants). FIG. 7d-f Demographic information of the TwinsUK study cohort (FIG. 1a, n=1,834 participants). FIG. 7a, b, d, e Distribution of the baseline Body Mass Index (BMI) (FIG. 7a, d) or age (FIG. 7b, c). n=821 (FIG. 7a, b; Female), 456 (FIG. 7a, b; Male), 1,774 (FIG. 7d, e; Female), 60 (FIG. 7d, c; Male) participants. The solid and dashed lines indicate the kernel density estimate and the mean of BMI (FIG. 7a, Female: 28.6 kg m-2; FIG. 7a, Male: 28.1 kg m-2; FIG. 7d, Female: 26.2 kg m-2; FIG. 7d, Male: 27.1 kg m-2) or age (FIG. 7b, Female: 47.6 years; FIG. 7b, Male: 44.7 years; FIG. 7c, Female: 61.4 years; FIG. 7e, Male: 62.0 years), respectively. FIG. 7c, f Composition of self-reported race (FIG. 7c) or ethnicity (FIG. 7f). The number in parentheses indicates the number of participants.

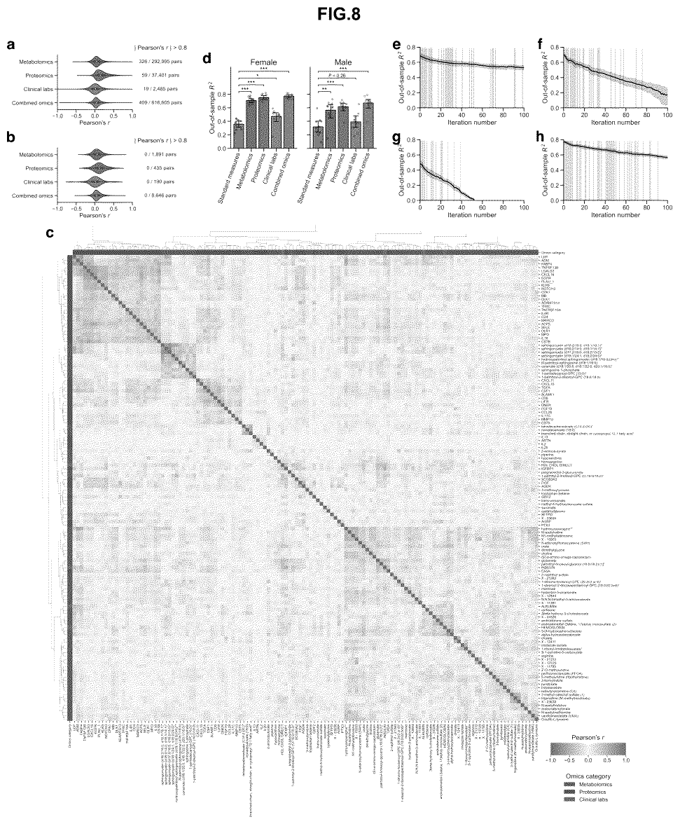

### FIG. 8

FIG. 8 shows quality check of the LASSO modeling. FIG. 8a, b Pairwise correlation of all plasma analytes (a; Metabolomics: 766 metabolites, Proteomics: 274 proteins, Clinical labs: 71 clinical laboratory tests, Combined omics: 1,111 analytes) or the analytes that were retained across all ten least absolute shrinkage and selection operator (LASSO) models (FIG. 8b; Metabolomics: 62 metabolites, Proteomics: 30 proteins, Clinical labs: 20 clinical laboratory tests, Combined omics: 132 analytes). Each violin is scaled to have same width between the omics categories and represents the kernel density distribution with boxplot (median: white point, [Q1, Q3]: box limits, [xmin, xmax]: whiskers, where Q1 and Q3 are the 1st and 3rd quartile values, and xmin and xmax are the minimum and maximum values in [Q1−1.5×IQR, Q3+1.5×IQR] (IQR: the interquartile range, Q3−Q1), respectively). FIG. 8c Hierarchical clustering and heatmap for the pairwise correlations of the analytes that were retained across all ten combined omics-based Body Mass Index (BMI) models (132 analytes: 77 metabolites, 51 proteins, 4 clinical laboratory tests). Of note, both upper and lower triangular sides of the symmetric matrix are visualized. FIG. 8d Model performance of each fitted BMI model with sex stratification. Out-of-sample R2 was calculated from each corresponding hold-out testing set. Standard measures: ordinary least squares (OLS) linear regression model with sex, age, triglycerides, high-density lipoprotein (HDL)-cholesterol, low-density lipoprotein (LDL)-cholesterol, glucose, insulin, and homeostatic model assessment for insulin resistance (HOMA-IR) as regressors. Data: mean with 95% confidence interval (CI), n=10 models. *Adjusted P<0.05, **adjusted P<0.01, ***adjusted P<0.001 in two-sided Welch's t-test with the Benjamini-Hochberg method across the eight (four comparisons×2 sexes) comparisons. Note that the sample size for modeling was different between female and male (Female: 821 participants, Male: 456 participants). FIG. 8e-h Transition of out-of-sample R2 in the LASSO-modeling iteration analysis for metabolomics (FIG. 8e), proteomics (FIG. 8f), clinical labs (FIG. 8g), or combined omics (FIG. 8h). At the end of each iteration, the variable that was retained across ten models and that had the highest absolute value for the mean of ten β-coefficients was removed from the input omic dataset. The iteration is highlighted with shading color when the removed analyte is the variable that was retained across all the original ten models. Data: mean with 95% CI, n=10 models.

### FIG. 9

FIG. 9 shows the restricted metabolomics-based BMI model predominantly maintained the characteristics of the original full model. FIG. 9a-c Comparison of the metabolomics-based Body Mass Index (MetBMI) model between the main analyses (Arivale cohort) and the validation analyses (TwinsUK cohort). Full version: least absolute shrinkage and selection operator (LASSO) model trained by all 766 metabolites in the Arivale dataset, Restricted version: LASSO model trained by the common 489 metabolites in the Arivale and TwinsUK datasets. FIG. 9a The number of the variables that were robustly retained across all ten MetBMI models. The number in square brackets indicates the number of the robustly retained metabolites that were derived from the common 489 metabolites. FIG. 9b Correlation of the mean of β-coefficients in the ten MetBMI models. Only the robustly retained metabolites in either full version (37 metabolites) or restricted version (74 metabolites) were analyzed. FIG. 9c Correlation of the MetBMI prediction. FIG. 9b, c The solid line is the ordinary least squares (OLS) linear regression line with 95% confidence interval (CI), and the dotted line in c is the value in full version=the value in restricted version. P: P-value of two-sided Pearson's correlation test. n=76 metabolites (b), 1,277 participants (FIG. 9c). FIG. 9d Correlation between the measured and predicted BMIs. The solid line is the OLS linear regression line with 95% CI, and the dotted line is measured BMI=predicted BMI. Standard measures: OLS linear regression model with sex, age, triglycerides, high-density lipoprotein (HDL)-cholesterol, low-density lipoprotein (LDL)-cholesterol, glucose, insulin, and homeostatic model assessment for insulin resistance (HOMA-IR) as regressors; Metabolomics: the restricted version of MetBMI model, corresponding to Metabolomics (restricted) in FIG. 1d; P: adjusted P-value of two-sided Pearson's correlation test with the Benjamini-Hochberg method across the four (two categories×two cohorts) tests. n=1,277 (Arivale), 1,834 (TwinsUK) participants.

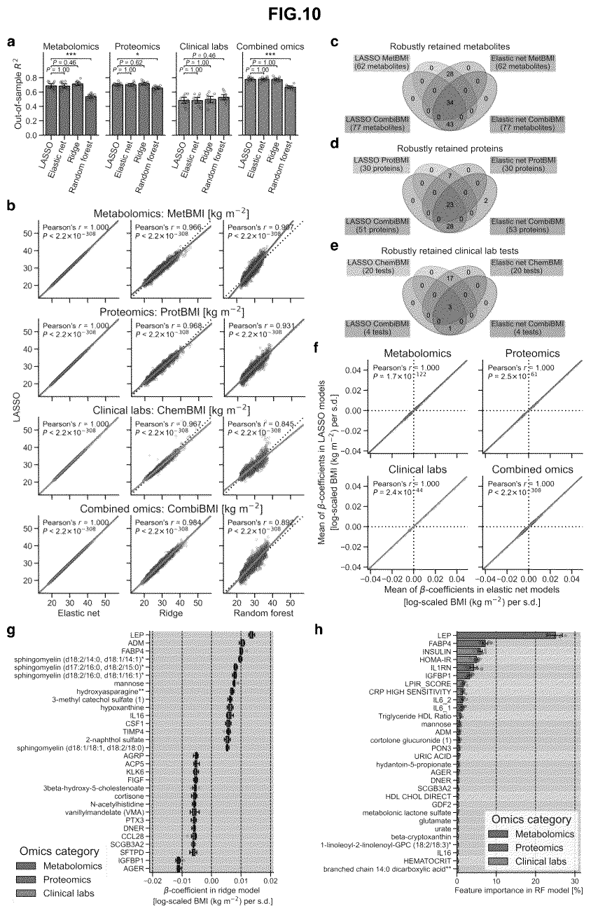

### FIG. 10

FIG. 10 shows omics-based BMI models were similar between LASSO and the other methods. FIG. 10a Model performance of each fitted Body Mass Index (BMI) model. Out-of-sample R2 was calculated from each corresponding hold-out testing set. Data: mean with 95% confidence interval (CI), n=10 models. *Adjusted P<0.05, ***Adjusted P<0.001 in two-sided Welch's t-test with the Benjamini-Hochberg method across the 12 (3 methods×4 categories) comparisons. FIG. 10b Correlation of the predicted BMI between LASSO and the other methods. The solid line is the ordinary least squares (OLS) linear regression line with 95% CI, and the dotted line is the value in LASSO=the value in the other method. P: adjusted P-value of two-sided Pearson's correlation test with the Benjamini-Hochberg method across the 12 (3 methods×4 categories) combinations. n=1,277 participants. FIG. 10c-f Comparison of the omics-based BMI model between LASSO and elastic net (EN) methods. FIG. 10c-e The number of the variables that were robustly retained across all ten LASSO or EN models. MetBMI: metabolomics-based BMI model, ProtBMI: proteomics-based BMI model, ChemBMI: clinical chemistries-based BMI model, CombiBMI: combined omics-based BMI model. FIG. 10f Correlation of the mean of β-coefficients in the ten omics-based BMI models. Only the robustly retained analytes in either LASSO models or EN models were analyzed. The solid line is the OLS linear regression line with 95% CI. P: adjusted P-value of two-sided Pearson's correlation test with the Benjamini-Hochberg method across the four categories. n=62 metabolites (Metabolomics), 30 proteins (Proteomics), 20 clinical laboratory tests (Clinical labs), 134 analytes (Combined omics). FIG. 10g The top 30 variables that had the highest absolute value for the mean of β-coefficients in the ten ridge CombiBMI models. β-coefficient was obtained from the fitted CombiBMI model with ridge regression. Data: median (center line), [Q1, Q3] (box limits), [xmin, xmax] (whiskers), where Q1 and Q3 are the 1st and 3rd quartile values, and xmin and xmax are the minimum and maximum values in [Q1−1.5×IQR, Q3+1.5×IQR] (IQR: the interquartile range, Q3−Q1), respectively; n=10 models. FIG. 10h The top 30 variables that had the highest mean of feature importance in the ten random forest (RF) CombiBMI models. The importance of a feature was calculated as the normalized total reduction of the mean squared error that was brought by the feature. Data: mean with 95% CI, n=10 models. FIG. 10g, h Each background color corresponds to the analyte category.

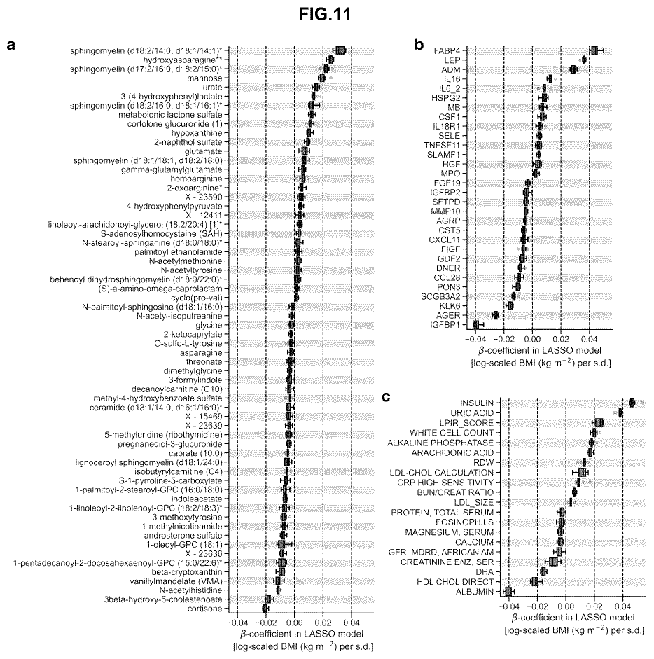

### FIG. 11

FIG. 11 shows variable diversity and contribution to the omics-based BMI model were different between omics categories. FIG. 11a-c The variables that were retained across all ten metabolomics-based (FIG. 11a), proteomics-based (FIG. 11b), or clinical labs-based (FIG. 11c) Body Mass Index (BMI) models (FIG. 11a: 62 metabolites, FIG. 11b: 30 proteins, FIG. 11c: 20 clinical laboratory tests). β-coefficient was obtained from the fitted omics-based BMI model with least absolute shrinkage and selection operator (LASSO) regression. Data: median (center line), [Q1, Q3] (box limits), [xmin, xmax] (whiskers), where Q1 and Q3 are the 1st and 3rd quartile values, and xmin and xmax are the minimum and maximum values in [Q1−1.5×IQR, Q3+1.5×IQR] (IQR: the interquartile range, Q3−Q1), respectively; n=10 models.

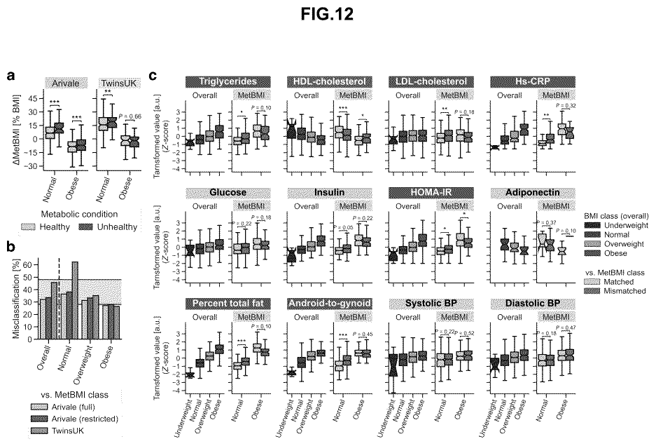

### FIG. 12

FIG. 12 shows the metabolic heterogeneity within the standard BMI classes was validated with the TwinsUK cohort. FIG. 12a Difference in ΔMetBMI (i.e., difference of the metabolomics-inferred Body Mass Index (MetBMI) from the measured BMI) between clinically-defined metabolic health conditions. Significance was assessed using ordinary least squares (OLS) linear regression with BMI, sex, and age as covariates, while adjusting multiple testing with the Benjamini-Hochberg method across the four (two BMI classes×two cohorts) regressions. For Arivale cohort, ancestry principal components (PCs) were also included in the covariates. MetBMI in Arivale was derived from the MetBMI model trained by the common 489 metabolites in the Arivale and TwinsUK datasets, corresponding to the restricted version in FIG. 9. FIG. 12b Misclassification rate of overall cohort or each BMI class against MetBMI class. Arivale (full): based on the full version of MetBMI model in FIG. 9 (i.e., the same with the corresponding ones in FIG. 3c), Arivale (restricted): based on the restricted version of MetBMI model in FIG. 9. Range of the previously reported misclassification rate is highlighted with pink background. Note that the underweight BMI class is not presented due to small sample size, but its misclassification rate was 100% against all omics-based BMI classes. FIG. 12c Difference in the obesity-related phenotypic measure between Matched and Mismatched groups in the TwinsUK cohort. Significance was assessed using OLS linear regression with BMI, sex, and age as covariates, while adjusting multiple testing with the Benjamini-Hochberg method across the 24 (2 BMI classes×12 measures) regressions. HDL: high-density lipoprotein, LDL: low-density lipoprotein, Hs-CRP: high-sensitivity C-reactive protein, Percent total fat: percentage of total fat in whole body, Android-to-gynoid: ratio of fat in android region to fat in gynoid region, HOMA-IR: homeostatic model assessment for insulin resistance, BP: blood pressure. FIG. 12a, c Data: median (center line), 95% confidence interval (CI) around median (notch), [Q1, Q3] (box limits), [xmin, xmax] (whiskers), where Q1 and Q3 are the 1st and 3rd quartile values, and xmin and xmax are the minimum and maximum values in [Q1−1.5×IQR, Q3+1.5×IQR] (IQR: the interquartile range, Q3−Q1), respectively; n=373 (FIG. 12a, Healthy in Normal of Arivale), 49 (FIG. 12a, Unhealthy in Normal of Arivale), 208 (FIG. 12a, Healthy in Obese of Arivale), 241 (FIG. 12a, Unhealthy in Obese of Arivale), 209 (FIG. 12a, Healthy in Normal of TwinsUK), 50 (FIG. 12a, Unhealthy in Normal of TwinsUK), 64 (FIG. 12a, Healthy in Obese of TwinsUK), 57 (FIG. 12a, Unhealthy in Obese of TwinsUK) participants (see Supplementary Data 6 for each sample size in FIG. 12c). *Adjusted P<0.05, **adjusted P<0.01, ***adjusted P<0.001.

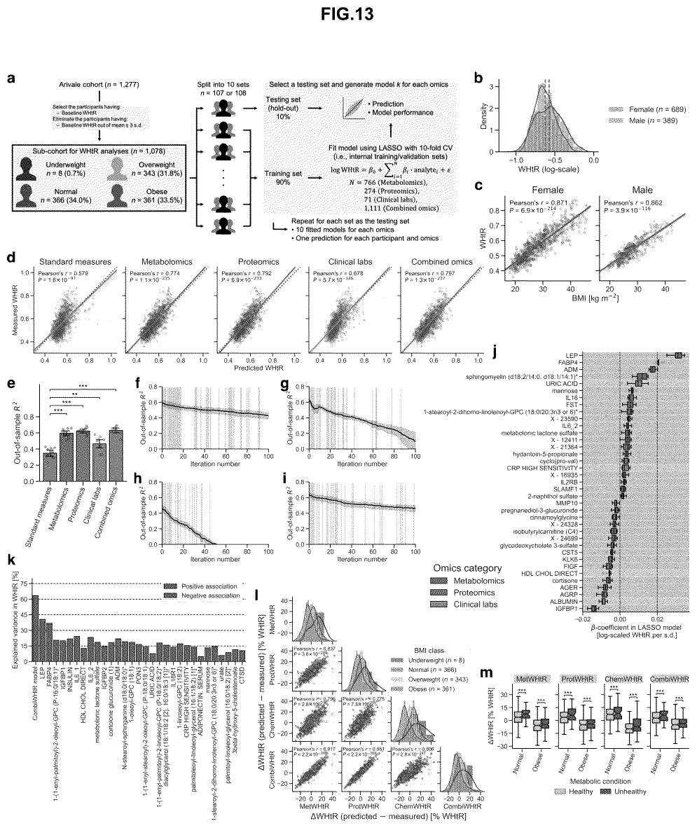

### FIG. 13

FIG. 13 shows omics-based WHtR models consistently supported the findings of omics-based BMI models. FIG. 13a Overview of study cohort and the omics-based waist-to-height ratio (WHtR) model generation. LASSO: least absolute shrinkage and selection operator, CV: cross-validation. FIG. 13b Distribution of the baseline WHtR. n=689 (Female), 389 (Male) participants. The solid and dashed lines indicate the kernel density estimate and the mean of WHtR (Female: 0.571, Male: 0.539 [raw scale]), respectively. FIG. 13c Correlation between the measured WHtR and Body Mass Index (BMI). The solid line is the ordinary least squares (OLS) linear regression line with 95% confidence interval (CI). P: adjusted P-value of two-sided Pearson's correlation test with the Benjamini-Hochberg method across the two sexes. n=689 (Female), 389 (Male) participants. FIG. 13d Correlation between the measured and predicted WHtRs. The solid line is the OLS linear regression line with 95% CI, and the dotted line is measured WHtR=predicted WHtR. Standard measures: OLS linear regression model with sex, age, triglycerides, high-density lipoprotein (HDL)-cholesterol, low-density lipoprotein (LDL)-cholesterol, glucose, insulin, and homeostatic model assessment for insulin resistance (HOMA-IR) as regressors; P: adjusted P-value of two-sided Pearson's correlation test with the Benjamini-Hochberg method across the five categories. n=1,078 participants. FIG. 13c Model performance of each fitted WHtR model. Out-of-sample R2 was calculated from each corresponding hold-out testing set. Data: mean with 95% CI, n=10 models. **Adjusted P<0.01, adjusted P<0.001 in two-sided Welch's t-test with the Benjamini-Hochberg method across the four comparisons. FIG. 13f-i Transition of out-of-sample R2 in the LASSO-modeling iteration analysis for metabolomics (FIG. 13f), proteomics (FIG. 13g), clinical labs (FIG. 13h), or combined omics (FIG. 13i). At the end of each iteration, the variable that was retained across ten models and that had the highest absolute value for the mean of ten β-coefficients was removed from the input omic dataset. The iteration is highlighted with shading color when the removed analyte is the variable that was retained across all the original ten models. Data: mean with 95% CI, n=10 models. FIG. 13j The variables that were retained across all ten combined omics-based WHtR (CombiWHtR) models (37 analytes: 18 metabolites, 15 proteins, and 4 clinical laboratory tests). β-coefficient was obtained from the fitted CombiWHtR model with LASSO regression. Each background color corresponds to the analyte category. Data: median (center line), [Q1, Q3] (box limits), [xmin, xmax] (whiskers), where Q1 and Q3 are the 1st and 3rd quartile values, and xmin and xmax are the minimum and maximum values in [Q1−1.5×IQR, Q3+1.5×IQR] (IQR: the interquartile range, Q3−Q1), respectively; n=10 models. FIG. 13k Univariate explained variance in WHtR by each analyte. WHtR was independently regressed on each of the analytes that were retained in at least one of the ten CombiWHtR models (288 analytes; Supplementary Data 9), using OLS linear regression with sex, age, and ancestry principal components (PCs) as covariates. Multiple testing was adjusted with the Benjamini-Hochberg method across the 289 regressions, including CombiWHtR model as reference. Among the analytes that were significantly associated with WHtR (212 analytes), only the top 30 significant analytes are presented with their univariate variances. FIG. 13l Difference of the omics-inferred WHtR from the measured WHtR (ΔWHtR). MetWHtR: metabolomics-inferred WHtR. ProtWHtR: proteomics-inferred WHtR, ChemWHtR: clinical chemistries-inferred WHtR, CombiWHtR: combined omics-inferred WHtR, P: adjusted P-value of two-sided Pearson's correlation test with the Benjamini-Hochberg method across the six combinations, FIG. 13n: the number of participants in each BMI class (total n=1,078 participants). The line in histogram panels indicates the kernel density estimate. FIG. 13m Difference in ΔWHtR between clinically-defined metabolic health conditions. Significance was assessed using OLS linear regression with WHtR, sex, age, and ancestry PCs as covariates, while adjusting multiple testing with the Benjamini-Hochberg method across the eight (two BMI classes×four omics categories) regressions. Data: each boxplot metric is the same with j, with the addition of 95% CI around median (notch); n=320 (Healthy in Normal), 42 (Unhealthy in Normal), 164 (Healthy in Obese), 197 (Unhealthy in Obese) participants. ***Adjusted P<0.001.

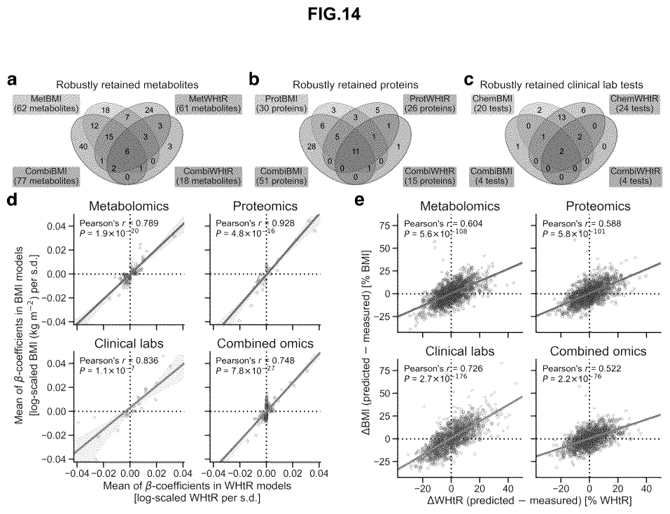

### FIG. 14

FIG. 14 shows predominant commonality with minor specificity was observed between the omics-based BMI and WHtR models. FIG. 14a-d Comparison of the omics-based least absolute shrinkage and selection operator (LASSO) model between Body Mass Index (BMI) and waist-to-height ratio (WHtR). FIG. 14a-c The number of the variables that were robustly retained across all ten LASSO models. MetBMI: metabolomics-based BMI model, MetWHtR: metabolomics-based WHtR model, ProtBMI: proteomics-based BMI model, ProtWHtR: proteomics-based WHtR model, ChemBMI: clinical chemistries-based BMI model, ChemWHtR: clinical chemistries-based WHtR model, CombiBMI: combined omics-based BMI model, CombiWHtR: combined omics-based WHtR model. FIG. 14d Correlation of the mean of β-coefficients in the ten LASSO models. Only the robustly retained analytes in either BMI models or WHtR models were analyzed. FIG. 14e Correlation between ABMI (i.e., difference of the omics-inferred BMI from the measured BMI) and ΔWHtR (i.e., difference of the omics-inferred WHtR from the measured WHtR). Only the participants having both BMI and WHtR were analyzed. FIG. 14d, e The solid line is the ordinary least squares (OLS) linear regression line with 95% confidence interval (CI). P: adjusted P-value of two-sided Pearson's correlation test with the Benjamini-Hochberg method across the four categories. n=92 metabolites (d, Metabolomics), 36 proteins (d, Proteomics), 26 clinical laboratory tests (FIG. 14d, Clinical labs), 146 analytes (FIG. 14d, Combined omics), 1,078 participants (FIG. 14c).

### Table Legends for Supplementary Data

Supplementary materials and supplementary data are available at medRxiv preprint doi: https://doi.org/10.1101/2022.01.20.22269601 (“Multiomic investigations of Body Mass Index reveal heterogeneous trajectories in response to a lifestyle intervention,” Kengo Watanabe et al., which is hereby incorporated by reference in its entirety for all purposes Supplementary data includes the following supplementary data:
Supplementary Data 1. A demographic summary of the study cohorts and statistical test summaries was generated for the independency of split sets.
Supplementary Data 2. Analytes of blood-measured omics and basic statistics of their baseline measurements were prepared from the Arivale and TwinsUK datasets.
Supplementary Data 3. β-coefficient estimates for the variables of the omics-based BMI models were generated related to FIG. 2 and FIGS. 8-11 and FIG. 14.
Supplementary Data 4. Relationships of the numeric physiological measures with the measured or omics-inferred BMI were examined. Regression analysis summary for the association between each of the 51 numeric physiological measures and the measured or omics-inferred BMI were generated, corresponding to FIG. 1c.
Supplementary Data 5. Relationships of the retained analytes in the omics-based BMI models with BMI were examined. Regression analysis summary for the association between BMI and each of the analytes that were retained in at least one of ten LASSO models were generated, corresponding to FIG. 2b-d.
Supplementary Data 6. Differences in phenotypic measures between the misclassification strata against the omics-inferred BMI class were investigated, and regression analysis summary for the difference in the obesity-related clinical blood marker, the BMI-associated numeric physiological feature, or the gut microbiome α-diversity metric between the misclassification strata against the omics-inferred BMI class were generated, corresponding to FIG. 3d,c, FIG. 4b and FIG. 12c.
Supplementary Data 7. Plasma analyte correlations modified by the baseline metabolic state and by lifestyle intervention were examined. An interaction analysis summary was prepared for the plasma analyte correlations modified by the baseline MetBMI and by days in program, corresponding to FIG. 6.
Supplementary Data 8. β-coefficient estimates were generated for the variables of the omics-based WHtR models, related to FIG. 13 and FIG. 14.
Supplementary Data 9. Relationships of the retained analytes in the omics-based WHtR models with WHtR were determined. A regression analysis summary for the association between WHtR and each of the analytes that were retained in at least one of ten LASSO models was generated, corresponding to FIG. 13k.
Supplementary Data 10. Statistical test summary were generated that included sample size, degrees of freedom, test statistic, (nominal) P-value, and adjusted P-value, corresponding to FIG. 1b-d, FIG. 3a, FIG. 3b, FIG. 4c-f and FIG. 8d, FIG. 9d, FIG. 10a, FIG. 10b, FIG. 10f, FIG. 12a, FIG. 13c-e, FIG. 13l, FIG. 13m, FIG. 14d, FIG. 14c.

### Example 2: Main Study Cohort

The main study cohort (Arivale cohort) was derived from 6,223 individuals who participated in a wellness program offered by a currently closed commercial company (Arivale Inc., Washington, USA) between 2015-2019. An individual was eligible for enrollment if the individual was over 18 years old, not pregnant, and a resident of any U.S. state except New York; participants were primarily recruited from Washington, California, and Oregon. The participants were not screened for any particular disease. During the Arivale program, each participant was provided personalized lifestyle coaching via telephone by registered dietitians, certified nutritionists, or registered nurses. This coaching was designed to improve the participant's health based on the combination of clinical laboratory tests, genetic predispositions, and published scientific evidence; e.g., reduction of sodium intake might be recommended to any participants with high blood pressure, but if they also had risk alleles indicating enhanced susceptibility to dietary sodium, this risk would be emphasized (see the previous report for more details). In this study, to compare the association between Body Mass Index (BMI) and host phenotypes across different omics, the original cohort was limited to the participants whose datasets contained (1) all main omic measurements (metabolomics, proteomics, clinical laboratory tests) from the same first blood draw, (2) a BMI measurement within +1.5 month from the first blood draw, and (3) genetic information (for using as covariates). Data that was eliminated were: (1) outlier participants whose baseline BMI was beyond ±3 s.d. from the mean in the baseline BMI distribution and (2) participants whose any of omic datasets contained more than 10% missingness in the filtered analytes (see the next section). The final Arivale cohort consisted of 1,277 (821 female and 456 male) participants (FIG. 1a), which exhibited consistent demographics (FIG. 7a-c, Supplementary Data 1) with the study cohorts defined in the previous Arivale studies. For the analyses of gut microbiome, sub-cohort was defined with the 702 (486 female and 216 male) participants from the Arivale cohort, who collected a stool sample within ±1.5 month from the first blood draw and did not use antibiotics in the last three months (FIG. 4a, Supplementary Data 1). For longitudinal analyses, sub-cohort was defined with the 608 (410 female and 198 male) participants from the Arivale cohort, whose datasets contained two or more time-series datasets for both BMI and omics during 18 months after enrollment (FIG. 5a, Supplementary Data 1). For the analyses of waist to-height ratio (WHtR), sub-cohort was defined with the 1,078 (689 female and 389 male) participants from the Arivale cohort, whose datasets contained the baseline WHtR measurement within +1.5 month from the first blood draw and within +3 s.d. from the mean in the baseline WHtR distribution (FIG. 13a, Supplementary Data 1).

### Example 3: Validation Cohort

The external cohort (TwinsUK cohort) was derived from 17,630 individuals who participated in the TwinsUK Registry, a British national register of adult twins31. Twins were recruited as volunteers by media campaigns without screening for any particular disease. The participants had two or more clinical visits for biological sampling between 1992-2022. In this study, to validate our findings in the Arivale cohort, the original cohort was limited to the participants whose datasets contained all measurements for metabolomics, BMI, and the obesity-related standard clinical measures (i.e., defined by triglycerides, high-density lipoprotein (HDL)-cholesterol, low-density lipoprotein (LDL)-cholesterol, glucose, insulin, and homeostatic model assessment for insulin resistance (HOMA-IR) in this study) from the same visit. Data that was also eliminated corresponded to: (1) outlier participants whose BMI was beyond ±3 s.d. from the mean in the overall BMI distribution and (2) participants whose metabolomic dataset contained more than 10% missingness in the filtered metabolites (see the next section). The final TwinsUK cohort consisted of 1,834 (1,774 female and 60 male) participants (FIG. 1a, FIG. 7d-f, Supplementary Data 1). For the analyses of gut microbiome, subcohort was defined with the 329 (307 female and 22 male) participants from the TwinsUK cohort, who collected a stool sample within +1.5 month from the clinical visit and did not use antibiotics at that time (FIG. 4a, Supplementary Data 1).

This study was conducted with de-identified data of the participants who had consented to the use of their anonymized data in research. All procedures were approved by the Western Institutional Review Board (WIRB) with Institutional Review Board (IRB) (Study Number: 20170658 at Institute for Systems Biology and 1178906 at Arivale) and by the TwinsUK Resource Executive Committee TREC) (Project Number: E1192).

### Example 4: Data Collections and Data Cleaning for Main Study Cohort

Multiomics data for the Arivale participants included genomics and longitudinal measurements of metabolomics, proteomics, clinical laboratory tests, gut microbiomes, wearable devices, and health/lifestyle questionnaires. Peripheral venous blood draws for all measurements were performed by trained phlebotomists at LabCorp (Laboratory Corporation of America Holdings, North Carolina, USA) or Quest (Quest Diagnostics, New Jersey, USA) service centers. Saliva to measure analytes such as diurnal cortisol and dehydroepiandrosterone (DHEA) was sampled by participants at home using a standardized kit (ZRT Laboratory, Oregon, USA). Likewise, stool samples for gut microbiome measurements were obtained by participants at home using a standardized kit (DNA Genotek, Inc., Ottawa, Canada).

### Genomics

DNA was extracted from each whole blood sample and underwent whole genome sequencing (1,257 participants) or single-nucleotide polymorphisms (SNP) microarray genotyping (20 participants). Genetic ancestry was calculated with principal components (PCs) using a set of ˜100,000 ancestry-informative SNP markers, as described previously. Polygenic risk scores (PRSs) were constructed using publicly available summary statistics from published genome wide association studies (GWAS), as described previously.

### Blood-Measured Omics

Metabolomics data was generated by Metabolon, Inc. (North Carolina, USA), using ultra high-performance liquid chromatography-tandem mass spectrometry (UHPLC-MS/MS) for plasma derived from each whole blood sample. Proteomics data was generated using proximity extension assay (PEA) for plasma derived from each whole blood sample with several Olink Target panels (Olink Proteomics, Uppsala, Sweden), and only the measurements with the Cardiovascular II, Cardiovascular III and Inflammation panels were used in this study since the other panels were not necessarily applied to all samples. All clinical laboratory tests were performed by LabCorp or Quest in a Clinical Laboratory Improvement Amendments (CLIA)-certified lab, and only the measurements by LabCorp were selected in this study to eliminate potential differences between vendors. In this study, the batch-corrected datasets with in-house pipeline were used, and metabolomic dataset was loge-transformed. In addition, analytes missing in more than 10% of the baseline samples were removed from each omic dataset, and observations missing in more than 10% of the remaining analytes were further removed. The final filtered metabolomics, proteomics, and clinical labs consisted of 766 metabolites, 274 proteins, 71 clinical laboratory tests, respectively (Supplementary Data 2).

### Gut Microbiome

Gut microbiome data was generated based on 16S amplicon sequencing of the V3+V4 region using a MiSeq sequencer (Illumina, Inc., California, USA) for DNA extracted from each stool sample, as previously described28. Briefly, the FASTQ files were processed using the mbtools workflow (https://github.com/Gibbons-Lab/mbtools) to remove noise, infer amplicon sequence variants (ASVs), and remove chimeras. Taxonomy assignment was performed using the SILVA ribosomal RNA gene database (version 132). In this study, the final collapsed ASV table across the samples consisted of 394, 341, 85, 45, 26, and 16 taxa for species, genus, family, order, class, and phylum, respectively. Gut microbiome α-diversity was

$$H = - \text{?} {p}_{i} ln {p}_{i}$$

$$\text{?} \text{indicates text missing or illegible when filed}$$

calculated at the ASV level using Shannon's index calculated by:
where p! is the proportion of a community i represented by ASVs, or using Chao1 diversity score calculated by:

$$\text{?} = \text{?} + \frac{\text{?}}{2 {n}_{2}}$$

$$\text{?} \text{indicates text missing or illegible when filed}$$

where Sobs is the number of observed ASVs, n1 is the number of singletons (ASVs captured once), and n2 is the number of doubletons (ASVs captured twice).

### Anthropometrics, Saliva-Measured Analytes, and Daily Physical Activity Measures

Anthropometrics including weight, height, and waist circumference (WC) and blood pressure were measured at the time of blood draw and also reported by participants, which generated diverse timing and number of observations depending on each participant. BMI and WHtR were simultaneously calculated from the measured anthropometrics with the weight divided by squared height [kg m-2] and the WC divided by height [unitless], respectively. Measurements of saliva samples were performed in the testing laboratory of ZRT Laboratory. Daily physical activity measures such as heart rate, moving distance, step count, burned calories, floors climbed, and sleep quality were tracked using the Fitbit wearable device (Fitbit, Inc., California, USA). To manage variations between days, monthly averaged data was used for these daily measures. In this study, the baseline measurement for these longitudinal measures was defined with the closest observation to the first blood draw per participant and data type, and each dataset was eliminated from analyses when its baseline measurement was beyond ±1.5 month from the first blood draw.

### Example 5: Data Collections and Data Cleaning for Validation Study Cohort

Data resource for the TwinsUK participants included longitudinal measurements of metabolomics, clinical laboratory tests, dual-energy X-ray absorptiometry (DXA), and health/lifestyle questionnaires. The necessary datasets for this study were provided by Department of Twin Research & Genetic Epidemiology (King's College London). In this study, after each provided dataset was cleaned as follows, the earliest visit among the visits from which all of metabolomics, BMI, and the standard clinical measures had been measured was defined as the baseline visit for each participant. As exception, the later visit among them was prioritized as the baseline visit if the participant had gut microbiome data within +1.5 month from the visit. Only the baseline visit measurements were analyzed.

### Blood-Measured Metabolomics

Metabolomics data was originally generated by Metabolon, Inc., using UHPLC-MS/MS for each serum sample. In this study, the provided median-normalized dataset was loge transformed. In addition, metabolites missing in more than 10% of the overall samples were removed from metabolomic dataset, and observations missing in more than 10% of the remaining metabolites were further removed. The final filtered metabolomics consisted of 683 metabolites.

### BMI

In this study, the BMI values that had been already calculated and included in the provided metabolomics data file were used.

### Standard Clinical Measures and Other Phenotypic Measures

In this study, because the provided phenotypic datasets contained multiple measurements for a phenotype even from a single visit of a participant (e.g., due to project difference, repeated measurements), multiple measurements were flattened into a single measurement for a phenotype per each participant's visit by taking the mean value. During this flattening step, difference in unit was properly adjusted, and the value indicating below detection limit was regarded as zero. HOMA-IR was calculated from the datasets of glucose, insulin, and fasting condition with the formula: HOMA-IR=fasting glucose [m mol L-1]×fasting insulin [mIU L-1]×22.5−1.

### Gut Microbiome

Gut microbiome data was originally generated based on whole metagenomic shotgun sequencing (WMGS) using a HiSeq 2500 sequencer (Illumina, Inc.) for DNA extracted from each stool sample45. In this study, the raw sequencing data was obtained from the National Center for Biotechnology Information (NCBI) Sequence Read Archive (SRA) project (PRJEB32731), and applied to a processing pipeline (https://github.com/Gibbons-Lab/pipelines). Briefly, the obtained FASTQ files were processed using the fastp (version 0.23.2) tool65 to filter and trim the reads, and taxonomic abundance was obtained using the Kraken 2 (version 2.1.2) and Bracken (version 2.6.0) tools66 with the Kraken 2 default database (based on NCBI RefSeq). The final collapsed taxonomic table across the samples consisted of 4,669, 1,225, 354, 167, 76, and 35 taxa for species, genus, family, order, class, and phylum, respectively.

### Example 6: Blood Omics-Based BMI and WHtR Models

For each Arivale baseline omic dataset, missing values were first imputed with a random forest (RF) algorithm using Python missingpy (version 0.2.0) library (corresponding to R MissForrest package). For sex-stratified models (Supplementary FIG. 2d), the datasets after imputation were divided into sex stratified datasets. Subsequently, the values in each omic dataset were standardized with Z-score using the mean and s.d. per analyte. Then, ten iterations of least absolute shrinkage and selection operator (LASSO) modeling with tenfold cross-validation (CV) were performed for the loge-transformed BMI or WHtR and each processed omic dataset, using LassoCV application programming interface (API) of Python scikit-learn (version 1.0.1) library. Training and testing (hold-out) sets were generated by splitting participants into ten sets with one set as a testing (hold-out) set and the remaining nine sets as a training set, and iterating all combinations over those ten sets; i.e., overfitting was controlled using tenfold CV with internal training and validation sets from each training set (FIG. 1a, FIG. 13a). Consequently, this procedure generated ten fitted sparse models for each omics category (Supplementary Data 3) and one single testing (hold-out) set-derived prediction from each omics category for each participant. The same modeling scheme while replacing LASSO with elastic net (EN), ridge, or RF was performed using Python scikit-learn ElasticNetCV, RidgeCV, or RandomForestRegressor-implemented GridSearchCV API, respectively. In this RF-modeling, the number of trees in the forest and the number of features were set as the hyperparameters to be decided through CV. For the standard measures-based models, the above modeling scheme was applied to ordinary least squares (OLS) linear regression with sex, age, triglycerides, HDL-cholesterol, LDL cholesterol, glucose, insulin, and HOMA-IR as regressors, using Python scikit-learn LinearRegression API. Of note, ten split sets were fixed among the omics categories and the modeling methods, and no significant difference in BMI, WHtR, sex, age, and ancestry PC1-5 among those ten sets was confirmed, using Pearson's χ2 test for categorical variable and Analysis of Variance (ANOVA) for numeric variable while adjusting multiple testing with the Benjamini-Hochberg method across the tested variables (Supplementary Data 1).

For the TwinsUK cohort, metabolomic dataset was applied to RF imputation and then each dataset of metabolomics and the standard clinical measures was applied to Z-score standardization, as well as the Arivale datasets. Utilizing the ten LASSO or OLS linear regression models that were fitted by the Arivale dataset, one single prediction was calculated from each processed dataset for each participant by taking the mean of ten predicted values. For metabolomics, ten metabolomics-based BMI (MetBMI) models were regenerated while restricting the input Arivale metabolomics to the common 489 metabolites in the Arivale and TwinsUK panels (FIG. 9).

For the LASSO-modeling iteration analysis (FIG. 9c-h, FIG. 13f-i), ten LASSO models were repeatedly generated with the above modeling scheme. At the end of each iteration, the variable that was retained across ten models and that had the highest absolute value for the mean of ten β-coefficients was removed from the input omic dataset.

For longitudinal predictions of the Arivale sub-cohort, one single prediction at a time point was calculated from each processed time-series omic dataset for each participant, utilizing the baseline LASSO model for which the participant was included in the baseline testing (hold-out) set. This was because (1) the baseline measurements were minimally affected by the personalized lifestyle coaching. (2) both count and time point of data collections were different among the participants, and (3) potential data leakage might be derived from the participant-measurement correspondence. For processing, each time-series omic dataset was applied to two-step RF imputation, where the baseline missingness was first imputed based on the baseline data structure and the remaining missingness was next imputed based on the overall data structure, and subsequently applied to Z-score standardization using the mean and s.d. in the baseline distribution.

Model performance was conservatively evaluated by the out-of-sample R2 that was calculated from each corresponding hold-out testing set in the Arivale cohort or from the external testing set in the TwinsUK cohort. Pearson's r between the measured and predicted values was calculated from the overall participants of the Arivale or TwinsUK cohort. Difference of the predicted value from the measured value (ΔMeasure; i.e., ΔBMI or ΔWHtR) was calculated with (the predicted value−the measured value)×(the measured value)−1×100 (i.e., the unit of ΔMeasure was [% Measure]). In the RF model, the importance of a feature was calculated as the normalized total reduction of the mean squared error that was brought by the feature.

### Example 7: Health Classification

Each participant was classified using each of the measured and omics-inferred BMIs based on the World Health Organization (WHO) international standards for BMI cutoffs (underweight: <18.5 kg m-2, normal: 18.5-25 kg m-2, overweight: 25-30 kg m-2, obese: ≥30 kg m-2)12. For the misclassification of BMI class against the omics-inferred BMI class, each participant was categorized into either Matched or Mismatched group when the measured BMI class was matched or mismatched to each omics-inferred BMI class, respectively.

For a clinically-defined metabolic health classification, the participants having two or more metabolic syndrome (MetS) risks of the National Cholesterol Education Program (NCEP) Adult Treatment Panel III (ATP III) guidelines were judged as the metabolically unhealthy group, while the other participants were judged as the metabolically healthy group. Concretely, the MetS risk components were (1) systolic blood pressure ≥130 mm Hg, diastolic blood pressure ≥85 mm Hg, or using antihypertensive medication, (2) fasting triglyceride level ≥150 mg dL-1, (3) fasting HDL cholesterol level <50 mg dL-1 for female and <40 mg dL-1 for male or using lipid-lowering medication, and (4) fasting glucose level ≥100 mg dL-1 or using antidiabetic medication. Only the participants who had all these information were assessed in the corresponding analyses (FIG. 3b; FIG. 12a, FIG. 13m).

### Example 8: Gut Microbiome-Based Models for Classifying Obesity

For the Arivale gut microbiome dataset, the whole ASV table (907 taxa from species to phylum) was preprocessed (i.e., positively shifted by one, loge-transformed, and standardized with Z-score using the mean and s.d. per taxon) and then applied to dimensionality reduction using PCA API of Python scikit-learn (version 1.0.1) library; the projected values onto the first 50 PCs (0.4-5.1% variance explained) were supplied as the input gut microbiome features. Two types of classifiers were trained on these gut microbiome features: one predicting whether an individual is obese BMI class and the other predicting whether an individual is obese MetBMI class. Both models were independently constructed through a fivefold iteration scheme of RF with fivefold CV, using Python scikit-learn RandomForestClassifier-implemented GridSearchCV API. In this RF-modeling, the number of trees in the forest and the number of features were set as the hyperparameters to be decided through CV. Training and testing (hold-out) sets were generated by splitting the participants of the normal and obese classes into five sets with one set as a testing (hold-out) set and the remaining four sets as a training set, and iterating all combinations over those five sets; i.e., overfitting was controlled using fivefold CV with internal training and validation sets from each training set (FIG. 4a). Consequently, this procedure generated five fitted classifiers for each BMI or MetBMI class and one single testing (hold-out) set-derived prediction from each classifier type for each participant. Note that the prediction included two types: either normal or obese class by a vote of the trees (i.e., binary prediction) and the mean probability of obese class among the trees.

For the TwinsUK gut microbiome dataset, the whole taxonomic table (6,526 taxa from species to phylum) was preprocessed and then applied to dimensionality reduction, as well as the Arivale dataset; the projected values onto the first 50 PCs (0.2-40.1% variance explained) were supplied as the input gut microbiome features. Then, the five obesity classifiers for each BMI or MetBMI class were generated as well as the above Arivale procedure, and one single testing (hold out) set-derived prediction from each classifier type was calculated for each participant (FIG. 4a).

Model performance of each classifier was conservatively evaluated using each corresponding hold-out testing set. Area under curve (AUC) in the receiver operator characteristic (ROC) curve and the average precision were calculated using the probability predictions, while sensitivity and specificity were calculated from confusion matrix using the binary predictions. The overall ROC curve and its AUC was calculated from all the participant's probability predictions, using R PROC (version 1.18.0) package.

### Example 9: Longitudinal Changes in the Measured and Omics-Inferred BMIs

A linear mixed model (LMM) was generated for each loge-transformed measured or omics-inferred BMI in the Arivale sub-cohort, following the previous approach. As fixed effects regarding time, linear regression splines with knots at 0, 6, 12, and 18 months were applied to days in program to fit time as a continuous variable rather than a categorical variable, because both count and time point of data collections were different among the participants. In addition to the linear regression splines of time as fixed effects, the LMM included sex, baseline age, ancestry PC1-5, and meteorological seasons as fixed effects (to adjust potential confounding effects) and random intercepts and random slopes of days in the program as random effects for each participant. Additionally, the same LMM for each measured or omics-inferred BMI was independently generated from each baseline BMI class stratified group. Of note, this stratified LMM was not generated from the underweight group because its sample size was too small for convergence. For comparing difference between the misclassification strata against the baseline MetBMI class, the above LMM while adding additional fixed effects, the categorical baseline misclassification of BMI class against MetBMI class (i.e., binary for Matched vs. Mismatched) and its interaction terms with the linear regression splines of time, was generated for each measured BMI or MetBMI from each baseline BMI class-stratified group. All LMMs were modeled using MixedLM API of Python statsmodels (version 0.13.0) library.

### Example 10: Plasma Analyte Correlation Network Analysis

Prior to the analysis, outlier values which were beyond ±3 s.d. from the mean in the Arivale subcohort baseline distribution were eliminated from the dataset per analyte, and seven clinical laboratory tests which became almost invariant across the participants were eliminated from analyses, allowing convergence in the following modeling. Per each analyte, values were converted with a transformation pipeline producing the lowest skewness (e.g., no transformation, the logarithm transformation for right skewed distribution, the square root transformation with mirroring for left skewed distribution) and standardized with Z-score using the mean and s.d.

Against 608,856 pairwise combinations of the analytes (766 metabolites, 274 proteomics, 64 clinical laboratory tests), generalized linear models (GLMs) for the baseline measurements of the Arivale sub-cohort (FIG. 5a; 608 participants) were independently generated with the Gaussian distribution and identity link function using glm API of Python statsmodels (version 0.13.0) library. Each GLM consisted of an analyte as dependent variable, another analyte and the baseline MetBMI as independent variables with their interaction term, and sex, baseline age, and ancestry PC1-5 as covariates. The analyte-analyte correlation pair that was significantly modified by the baseline MetBMI was obtained based on the β-coefficient (two-sided t-test) of the interaction term between independent variables in GLM, while adjusting multiple testing with the Benjamini-Hochberg method (false discovery rate (FDR)<0.05).

Against the significant 100 pairs from the GLM analysis (82 metabolites, 33 proteins, and 16 clinical laboratory tests; Supplementary Data 7), generalized estimating equations (GEEs) for the longitudinal measurements of the metabolically obese group (i.e., the baseline obese MetBMI class; 182 participants) were independently generated with the exchangeable covariance structure using Python statsmodels GEE API. Each GEE consisted of an analyte as dependent variable, another analyte and days in the program as independent variables with their interaction term, and sex, baseline age, ancestry PC1-5, and meteorological seasons as covariates. The analyte-analyte correlation pair that was significantly modified by days in the program was obtained based on the β-coefficient (two sided t-test) of the interaction term between independent variables in GEE, while adjusting multiple testing with the Benjamini-Hochberg method (FDR<0.05).

### Example 11: Statistical Analysis

All data preprocessing and statistical analyses were performed using Python NumPy (version 1.18.1 or 1.21.3). pandas (version 1.0.3 or 1.3.4), SciPy (version 1.4.1 or 1.7.1) and statsmodels (version 0.11.1 or 0.13.0) libraries, except for using R pROC (version 1.18.0) package for DeLong's test. All statistical tests were performed using a two-sided hypothesis. In all cases of multiple testing, P-value was adjusted with the Benjamini-Hochberg method. Of note, because some hypotheses were not completely independent (e.g., between combined omics and each individual omics; between glucose, insulin, and HOMA-IR), this simple P-value adjustment was regarded as a conservative approach. Significance was based on P<0.05 for single testing and FDR<0.05 for multiple testing. Test summaries (e.g., sample size, degrees of freedom, test statistic, exact P-value) are found in Supplementary Data 4, 5, 6, 7, 9, and 10.

Correlations (FIG. 1b, FIG. 3a; FIG. 9b-d; FIG. 10b,f; FIG. 13c,d,l; FIG. 14d,e) were independently assessed using Pearson's correlation test (Python SciPy pearsonr API), with the P840 value adjustment if multiple testing. Comparisons of model performance (FIG. 1c,d; FIG. 4d,f; FIG. 8d; FIG. 10a; FIG. 13e) were independently assessed using Welch's t-test (Python statsmodels ttest_ind API), with the P-value adjustment if multiple testing. Comparison of overall ROC curves (FIG. 4c,e) was assessed using unpaired DeLong's test.

In all regression analyses, only the baseline datasets were used, and, unless otherwise specified, all numeric variables were centered and scaled in advance. For the Arivale datasets of anthropometrics, saliva-measured analytes, daily physical activity measures, and PRSs, (1) outlier values which were beyond ±3 s.d. from the mean in the cohort distribution were eliminated from the dataset per variable. (2) variables which became almost invariant across the participants were eliminated from the datasets, (3) values were converted with a transformation pipeline producing the lowest skewness (e.g., no transformation, the logarithm transformation for right skewed distribution, the square root transformation with mirroring for left skewed distribution), and (4) the transformed values were standardized with Z-score using the mean and s.d.; these preprocessed 51 variables were used as the numeric physiological features (Supplementary Data 4). As well, the Arivale datasets of the obesity-related clinical blood markers (i.e., selected clinical labs: Supplementary Data 6) and the TwinsUK datasets of the obesity-related phenotypic measures (Supplementary Data 6) were preprocessed. For gut microbiome α-diversity metrics, the number of observed ASVs and Chao1 index were converted with square root transformation while Shannon's index was converted with square transformation, and then these transformed values were standardized with Z-score using the mean and s.d. Relationships of the numeric physiological features with the measured or omics-inferred BMI (FIG. 1e, Supplementary Data 4) were independently assessed using each OLS linear regression model with the (unstandardized) loge-transformed measured or omics-inferred BMI as dependent variable, a feature as independent variable, and sex, age, and ancestry PC1-5 as covariates, while adjusting multiple testing across the 255 (51 features×5 BMI types) regressions. Relationships between BMI and the analytes that were retained in at least one of ten LASSO models (FIG. 2b-d, Supplementary Data 5) were independently assessed using each OLS linear regression model with the (unstandardized) loge-transformed BMI as dependent variable, an analyte as independent variable, and sex, age, and ancestry PC1-5 as covariates, while adjusting multiple testing across the 210 (FIG. 2b), 75 (FIG. 2c), or 42 (FIG. 2d) regressions. In this regression analysis, a model including the omics inferred BMI as independent variable was also assessed as reference. Differences in ΔMeasure (i.e., ABMI or ΔWHtR) between clinically-defined metabolic health conditions (FIG. 3b; FIG. 12a; FIG. 13m) were independently assessed using each OLS linear regression model with ΔMeasure as dependent variable, metabolic condition (i.e., Healthy vs. Unhealthy) as categorical independent variable, and Measure, sex, age, and ancestry PC1-5 as covariates, while adjusting multiple testing across the eight (two BMI classes×four omics categories; FIG. 3b. FIG. 13m) or four (two BMI classes×two cohorts; FIG. 12a) regressions. Differences in the obesity related clinical blood markers, the BMI-associated numeric physiological features, or the gut microbiome α-diversity metrics between the misclassification strata against the omics-inferred BMI class (FIG. 3d, 3e, 4b; FIG. 12c) were independently assessed using each OLS linear regression model with a marker, feature, or metric as dependent variable, misclassification (i.e., Matched vs. Mismatched) as categorical independent variable, and BMI, sex, age, and ancestry PC1-5 as covariates, while adjusting multiple testing across the 40 (2 BMI classes×2 omics categories×10 markers; FIG. 3d), 216 (2 BMI classes×4 omics categories×27 features; FIG. 3e), 24 (2 BMI classes×4 omics categories×3 metrics; FIG. 4b), or 24 (2 BMI classes×12 measures; FIG. 12c) regressions. In the above regression analyses for the TwinsUK cohort, ancestry PCs were eliminated from the covariates due to data availability.

### Example 12: Data Visualization

Results were visualized using Python matplotlib (version 3.4.3) and seaborn (version 0.11.2) libraries, except for the plasma analyte correlation network. Data were summarized as the mean with 95% confidence interval (CI) or the boxplot (median: center line; 95% CI around median: notch; [Q1, Q3]: box limits; [xmin. xmax]: whiskers, where Q1 and Q3 are the 1st and 3rd quartile values and xmin, and xmax are the minimum and maximum values in [Q1−1.5×IQR, Q3+1.5×IQR] (IQR: the interquartile range, Q3−Q1), respectively), as indicated in each figure legend. For presentation purpose, CI was simultaneously calculated during visualization using Python seaborn barplot or boxplot API with default setting (1,000 times bootstrapping or a Gaussian-based asymptotic approximation, respectively). The OLS linear regression line with 95% CI was simultaneously generated during visualization using Python seaborn regplot API with default setting (1,000 times bootstrapping). The plasma analyte correlation network was visualized with a circos plot using R circlize (version 0.4.15) package.

### Example 13: Data and Code Availability

The de-identified Arivale datasets used in this study were provided by the Institute of Systems Biology (http://isbscience.org). The de-identified TwinsUK datasets used in this study were provided by Department of Twin Research & Genetic Epidemiology (King's College London) (Project Number: E1192) (http://twinsuk.ac.uk/907 resources-for-researchers/access our-data/). Code used in this study can be accessed on GitHub (https://github.com/PriceLab/Multiomics-BMI).

### Example 14: Plasma Multiomics Captured 48-78% of the Variance in BMI

To investigate the molecular phenotypic perturbations associated with obesity, a study cohort was selected of 1.277 adults who participated in a scientific wellness program (Arivale) (20,24-29) and whose datasets included coupled measurements of plasma metabolomics, proteomics, and clinical laboratory tests from the same blood draw (FIG. 1a; see above methods). This study design allowed us to directly investigate the similarities and differences between omics platforms with regards to how they reflected the physiological health state of each individual across the BMI spectrum. This cohort was characteristically female (64.3%), middle-aged (mean±s.d.: 46.6±10.8 years), and white (69.7%) (Supplementary FIG. 1a-c; Supplementary Data 1). Based on the World Health Organization (WHO) international standards for BMI cutoffs (underweight: <18.5 kg m-2, normal: 18.5-25 kg m-2, overweight: 25-30 kg m-2, obese: ≥30 kg m-2), the baseline BMI prevalence was similar among normal, overweight, and obese classes, while only 0.8% of participants were in the underweight class (underweight: 10 participants (0.8%), normal: 426 participants (33.4%), overweight: 391 participants (30.6%), obese: 450 participants (35.2%)).

Leveraging the baseline measurements of plasma molecular analytes (766 metabolites, 274 proteins, and 71 clinical laboratory tests; Supplementary Data 2), machine learning models were trained to predict baseline BMI (i.e., not forecast a future outcome but calculate an out-of-sample outcome) for each of the omics platforms (metabolomics, proteomics, and clinical labs) or in combination (combined omics of all metabolomics, proteomics, and clinical labs): metabolomics-based, proteomics-based, clinical labs (chemistries)-based, and combined omics-based BMI (MetBMI, ProtBMI, ChemBMI, and CombiBMI, respectively) models. To address multicollinearity among the analytes (FIG. 8a) and to obtain predictions for all participants, a tenfold iteration scheme of the least absolute shrinkage and selection operator (LASSO) algorithm with tenfold cross-validation (CV) was applied (FIG. 1a; see above methods). This approach generated ten fitted sparse models for each omics category (Supplementary Data 3) and one single testing (hold-out) set-derived prediction from each omics category for each participant. The resulting models retained (i.e., assigned non-zero ß-coefficient to) 62 metabolites, 30 proteins, 20 clinical laboratory tests, and 132 analytes across all ten MetBMI, ProtBMI. ChemBMI, and CombiBMI models, respectively, which exhibited low collinearity (FIG. 8b, c) as expected from the LASSO algorithm (30). In contrast to a model including obesity-related standard clinical measures (i.e., ordinary least squares (OLS) linear regression model with sex, age, triglycerides, high-density lipoprotein (HDL)-cholesterol, low-density lipoprotein (LDL)-cholesterol, glucose, insulin, and homeostatic model assessment for insulin resistance (HOMA-IR) as regressors; StandBMI model), each omics-based model demonstrated significantly higher performance in BMI prediction, ranging from out-of-sample R2=0.48 (ChemBMI) to 0.70 (ProtBMI) compared to 0.37 (StandBMI) (FIG. 1b, c). The CombiBMI model exhibited the best performance in BMI prediction (out-of-sample R2=0.78; FIG. 1c), but the variances explained were not completely additive, suggesting that, although there is a considerable overlap in the signal detected by each omics platform, different omic measurements still contain non-redundant information regarding BMI. Additionally, these results were consistent in sex-stratified models, with the exception of male ChemBMI model that tended to exhibit higher performance than StandBMI model without statistical significance (FIG. 8d).

To confirm the generalizability of our results, an external cohort of 1,834 adults from the TwinsUK registry was investigated. The cohort's datasets included serum metabolomics and the aforementioned standard clinical measures (FIG. 1a; see above methods). This external cohort was demographically distinct from the Arivale cohort (FIG. 7d-f; Supplementary Data 1); the TwinsUK cohort was overwhelmingly female (96.7%), senior (mean±s.d.: 61.4±9.0 years), and white (99.2%), and consisted of 15 (0.8%), 779 (42.5%), 706 (38.5%), and 334 (18.2%) participants in the underweight, normal, overweight, and obese BMI classes, respectively. To manage the differences in the metabolomics panels. MetBMI models were regenerated in the Arivale cohort, while restricting the metabolomic features to an overlapping set of 489 metabolites between the Arivale and TwinsUK panels (called restricted model). Although 25 of the retained metabolites in the original MetBMI models were replaced with other metabolites due to their absences in the restricted panel, 35 of the remaining 37 metabolites were consistently retained across the restricted MetBMI models (FIG. 9a). Moreover, β-coefficients for the retained metabolites and MetBMI predictions for the Arivale cohort were consistent between the original and restricted models (FIG. 9b, c). BMI predictions for the TwinsUK cohort were calculated using the StandBMI and restricted MetBMI models that were fitted to the Arivale datasets. The restricted MetBMI model exhibited a lower absolute performance on the TwinsUK cohort compared to the Arivale cohort, but a significantly higher performance than StandBMI model (out-of-sample R2=0.29 (MetBMI), −0.13 (StandBMI); FIG. 1d, FIG. 9d), confirming that blood metabolomics generally captures BMI better than the standard clinical measures.

BMI has been reported to be associated with multiple anthropometric and clinical measures, such as waist circumference (WC), blood pressure, sleep quality, and several polygenic risk scores (PRSs). Thus, the association between the omics-inferred BMI and each of the available numeric physiological measures was examined (see above methods; Supplementary Data 4). Among the 51 assessed features, measured BMI was significantly associated with 27 features (false discovery rate (FDR)<0.05) including daily physical activity measures from wearable devices, waist-to-height ratio (WHtR), blood pressure, and BMI PRS (FIG. 1e). With minor differences in effect sizes, these BMI associated features were concordantly associated with each omics-inferred BMI (FIG. 1e), indicating that the omics-inferred BMIs primarily maintain the characteristics of classical BMI in terms of anthropometric, genetic, lifestyle, and physiological associations.

### Example 15: Omics-Based BMI Estimates Captured the Variation in BMI Better than any Single Analyte

Because our LASSO linear regression model showed comparable performance to elastic net (EN) and ridge linear regression models and a non-linear random forest (RF) regression model (FIG. 10a, b), and because LASSO model β-coefficients are generally easier to be interpreted, the LASSO models were used as a focus. However, the LASSO algorithm randomly retains variables from highly collinear groups, and sets β-coefficients of the other variables to zero. To confirm the robustness of the variable selection process, the LASSO modeling was iterated while removing the strongest analyte (i.e., the analyte that had the highest absolute value for the mean of the ten β-coefficients) from the input omic dataset at the end of each iteration. If a variable is indispensable for a model, the performance should largely decrease after removing it. In all omics categories, a steep decay in the out-of-sample R2 was observed in the first 5-9 iterations (FIG. 8e-h), suggesting that, at least, the top 5-9 variables that had the highest absolute β-coefficient values in the original LASSO models were indispensable for predicting BMI. Interestingly, the overall slope of R2 in MetBMI model decayed more gradually compared to ProtBMI and ChemBMI models (FIG. 8e-g), implying that metabolomics data contain more redundant information about BMI than the other omics data. Although larger number of metabolites in the input dataset might be a plausible explanation, the proportion of the variables that were robustly retained across all ten LASSO models (FIG. 11) to the variables that were retained in at least one of the ten LASSO models was lower in MetBMI model compared to ProtBMI and ChemBMI models (MetBMI: 62/209 metabolites ˜29.7%, ProtBMI: 30/74 proteins ˜40.5%, ChemBMI. 20/41 clinical laboratory tests ˜48.9%), confirming the higher level of redundancy within metabolomics data. Nevertheless, even though the high redundancy, metabolites still constituted 58% of the 132 analytes that were retained across all ten CombiBMI models (77 metabolites, 51 proteins, 4 clinical laboratory tests; FIG. 2a), suggesting that each of the omics categories possesses unique information about BMI. The strongest predictors in CombiBMI model were primarily proteins; e.g., analytes having the mean absolute β-coefficient >0.02 (i.e., affecting more than ˜2% BMI in prediction per 1 s.d. of its change, according to the Taylor/Maclaurin series: eβ≈1+β when β<<1) were leptin (LEP), adrenomedullin (ADM), and fatty acid-binding protein 4 (FABP4) as the positive predictors and insulin-like growth factor-binding protein 1 (IGFBP1) and advanced glycosylation end-product specific receptor (AGER; also described as receptor of AGE, RAGE) as the negative predictors. Note that these strongest proteins were consistent in the EN models (FIG. 10c-f) and had high importance in the ridge and RF models (FIG. 10g, h).

At the same time, the existence of these strong and consistently-retained predictors in the omics-based BMI models implied that a single analyte might be a suitable biomarker to predict BMI. To address this possibility. BMI was regressed independently on each of the analytes that were retained in at least one of the ten LASSO models (MetBMI: 209 metabolites. ProtBMI: 74 proteins, ChemBMI: 41 clinical laboratory tests; Supplementary Data 5). Among the analytes that were significantly associated with BMI (180 metabolites. 63 proteins, 30 clinical laboratory tests), only LEP, FABP4, and interleukin 1 receptor antagonist (IL1RN) exhibited over 30% of the explained variance in BMI by themselves (FIG. 2b-d), with a maximum of 37.9% variance explained (LEP). In contrast. MetBMI. ProtBMI, and ChemBMI models explained 68.9%, 70.6%, and 48.8% of the variance in BMI, respectively. Moreover, even upon eliminating several strong predictor analytes such as LEP and FABP4 from the omic datasets, the models still explained more variance in BMI than any single analyte (FIG. 8e-h). These results indicate that the multiomic BMI prediction models explain a larger portion of the variation in BMI than any single analyte, and highlight the multivariate perturbation of blood analytes across all platforms with increasing BMI.

### Example 16: Metabolic Heterogeneity was Responsible for the High Rate of Misclassification within the Standard BMI Classes

While the omics-inferred BMIs showed the similar phenotypic associations as the measured BMI (FIG. 1e), the difference of the predicted BMI from the measured BMI (ΔBMI) was highly correlated among the omics-based BMI models, ranging from Pearson's r=0.64 (ChemBMI vs. CombiBMI) to 0.83 (ProtBMI vs. CombiBMI) (FIG. 3a). In other words, the different omics consistently detected deviation of the omics-inferred BMI from the measured BMI per individual, implying that this deviation stemmed from a true biological signal of a perturbed physiological state rather than from noise or modeling artifacts. Actually, when individuals in the normal and obese BMI classes (defined by the WHO international standards) were subdivided by a clinical definition of metabolic health (i.e., defining metabolically unhealthy if having two or more MetS risks of the National Cholesterol Education Program (NCEP) Adult Treatment Panel III (ATP III) guidelines; see above methods), ABMI was significantly higher in MUNW and MUO groups compared to metabolically healthy, normal-weight (MHNW) and MHO groups, respectively, for all omics categories (FIG. 3b), suggesting that the deviations of model predictions are related to metabolic health.

Nevertheless, there has been no universally accepted definition of metabolic health. Thus, given the high interpretability and intuitiveness of the omics-inferred BMI, a potential application was explored: using the omics-inferred BMI (instead of the measured BMI) for improved classification of both obesity and metabolic health with the WHO international standards. Each participant was classified using each of the measured and omics-inferred BMIs based on the standard BMI cutoffs, and categorized into either Matched or Mismatched group when the measured BMI class was matched or mismatched to each omics-inferred BMI class, respectively. The misclassification rate against the omics-inferred BMI class was ˜30% across all omics categories and BMI classes (FIG. 3c), consistent with the previously reported misclassification rates about the cardiometabolic health classification. Relationships between this omics-based misclassification were examined within normal or obese BMI class and the obesity-related clinical blood markers (Supplementary Data 6), including triglycerides, HDL-cholesterol, LDL-cholesterol, high-sensitivity C-reactive protein (hs-CRP), glucose, insulin, HOMA-IR, glycated hemoglobin A1c (HbA1c), adiponectin, and vitamin D. Because ChemBMI and CombiBMI models were not independent of these markers, only the misclassification against MetBMI or ProtBMI class was examined in this analysis. The Mismatched group of normal BMI class exhibited significantly higher values of the markers that are positively associated with BMI (+BMI), such as triglycerides, hs-CRP, glucose, and HOMA-IR, and significantly lower values of the markers that are negatively associated with BMI (−BMI), such as HDL-cholesterol and adiponectin, compared to the Matched group of normal BMI class (FDR<0.05; FIG. 3d). These patterns suggest that the participant misclassified into the normal BMI class possesses less healthy molecular profiles as similarly as the individual with overweight or obesity, corresponding to the individual with MUNW phenotype. Conversely, the Mismatched group of obese BMI class exhibited significantly lower and higher values of the positively and negatively BMI associated markers, respectively, compared to the Matched group of obese BMI class (FDR<0.05; FIG. 3d), suggesting that the participant misclassified as obese BMI class has healthier blood signatures, more similarly to the individual with overweight or normal-weight, corresponding to the individual with MHO phenotype. Likewise, the 27 BMI-associated numeric physiological features (FIG. 1e, Supplementary Data 6) were re-examined, and found the concordant pattern of significant phenotypic differences between Matched and Mismatched groups in WHtR (+BMI), heart rate (+BMI), blood pressure (+BMI), and daily physical activity measures (−BMI) (FDR<0.05; FIG. 3c). Importantly, there was no difference in BMI PRS (+BMI) between Matched and Mismatched groups (FIG. 3e), implying that lifestyle or environmental factors, rather than genetic risk, is likely involved in the discordance between the measured and omics-inferred BMIs. Furthermore, these findings were validated and expanded in the TwinsUK cohort: ΔMetBMI was significantly higher in the metabolically unhealthy group compared to the metabolically healthy group within the normal BMI class (FIG. 12a); the misclassification rate against MetBMI class was much higher (>60%) in the normal BMI class but ˜30% in the others (FIG. 12b); the concordant phenotypic differences between Matched and Mismatched groups were significantly observed in triglycerides (+BMI), HDL-cholesterol (−BMI), LDL-cholesterol (+BMI), hs-CRP (+BMI), and HOMA-IR (+BMI) (FDR<0.05; FIG. 12c). Remarkably, while DXA measurements were not performed in the Arivale cohort, the percentage of total fat in whole body (+BMI) and the ratio of fat in android region to fat in gynoid region (+BMI) were significantly higher in Mismatched group compared to Matched group within the normal BMI class of the TwinsUK cohort (FDR<0.05; FIG. 12c). Taken together, these results suggest that the omics-based BMI models can identify heterogeneous metabolic health states which are not captured by the measured BMI with the standard BMI cutoffs.

### Example 17: Metabolomics-Inferred BMI Reflected Gut Microbiome Profiles Better than BMI

The gut microbiome has been shown to causally affect host obesity phenotypes in a mouse model and humans with obesity generally exhibit lower bacterial α-diversity (i.e., the species richness and/or evenness of an ecological community). However, certain meta-analyses of human case-control studies suggest an inconsistent relationship between the gut microbiome and obesity. Given our previous finding that the association between blood metabolites and bacterial diversity is dependent on BMI and the current finding that the omics-based BMI models capture heterogeneous metabolic health states (FIG. 3), a hypothesis was that MetBMI represents gut microbiome α-diversity better than the measured BMI. For the 702 Arivale participants who had both stool-derived gut microbiome and blood omic datasets (FIG. 4a; see above methods), relationships between gut microbiome α-diversity (the number of observed species, Shannon's index, and Chao1 index) and the omics-based BMI misclassification were examined. Matched and Mismatched groups against MetBMI class showed significant differences in all α-diversity metrics within both normal and obese BMI classes (FIG. 4b), with the concordant pattern to the clinical markers and BMI-associated features (−BMI; e.g., HDL-cholesterol; FIG. 3d, c), implying that the MetBMI class reflects bacterial diversity better than BMI class. Interestingly, the misclassification against the other omics categories did not show these significant differences for all α-diversity metrics and both BMI classes (FIG. 4b), consistent with our previous observation that plasma metabolomics showed a much stronger correspondence to gut microbiome structure than either proteomics or clinical labs.

We further examined the predictive power of gut microbiome profiles for MetBMI. For each of the measured BMI and MetBMI classes, models classifying individuals into normal class versus obese class based on gut microbiome 16S rRNA gene amplicon sequencing data were generated, using a fivefold iteration scheme of the RF algorithm with fivefold CV (FIG. 4a; see above methods). Compared to the classifier for the measured BMI class, the classifier for MetBMI class showed significantly larger area under curve (AUC) in the receiver operator characteristic (ROC) curve in the Arivale cohort (AUC=0.66 (BMI), 0.75 (MetBMI); FIG. 4c), with significantly higher sensitivity and precision (FIG. 4d). Moreover, by applying the same scheme to the stool-derived whole metagenomic shotgun sequencing (WMGS) data of the 329 TwinsUK participants (FIG. 4a; see above methods), the hypothesis that the gut microbiome-based obesity classifier for MetBMI class significantly outperformed the classifier for the measured BMI class in the TwinsUK cohort (AUC=0.57 (BMI). 0.75 (MetBMI); FIG. 4e, f) was examined. Note that these classifiers were regenerated for the TwinsUK cohort (instead of using the classifiers that were fitted to the Arivale dataset; FIG. 4a) due to the difference in sequencing methods (amplicon sequencing vs. WMGS), while considering that the TwinsUK participants' MetBMIs were predicted from the Arivale-fitted MetBMI models (FIG. 1a). Altogether, these findings suggest that, although other factors (e.g., dietary intake 19) may be involved, MetBMI has a stronger correspondence to gut microbiome features than the standard BMI.

### Example 18: Metabolic Health of the Metabolically Obese Group was Substantially Improved Following a Healthy Lifestyle Intervention

In the Arivale program, healthy lifestyle coaching was provided to all participants, resulting in clinical improvement across multiple measures of health. This coaching intervention was personalized for each participant to improve the participant's health based on the combination of clinical laboratory tests, genetic predispositions, and published scientific evidence, and administered via telephone by registered dietitians, certified nutritionists, or registered nurses (see above methods and a previous report). To investigate the longitudinal changes in omic profiles during the program, a sub-cohort of 608 participants was defined based on the available longitudinal measurements (FIG. 5a; see above methods). Given the participant-dependent variability in both count and time point of data collections, the average trajectory of each measured or omics-inferred BMI in the Arivale sub-cohort was estimated using a linear mixed model (LMM) with random effects for each participant (see above methods). Consistent with the previous analysis, the mean BMI estimate for the overall cohort decreased during the program (FIG. 5b). The decrease of MetBMI was larger than that of measured BMI, while the decrease of ProtBMI was minimal and even smaller than that of measured BMI (FIG. 5b), suggesting that plasma metabolomics is highly responsive to the lifestyle intervention in the short term, while proteomics (measured from the same blood draw) is more resistant to change during the same intervention period. Subsequently, LMMs were generated with the baseline BMI class stratification, and confirmed that a significant decrease in the mean BMI estimate was observed in the overweight and obese BMI classes, but not in the normal BMI class (FIG. 5c). Concordantly, the mean estimates of ProtBMI and ChemBMI exhibited negative changes over time in the overweight and obese BMI classes, but not in the normal BMI class (FIG. 5c). In contrast, the mean estimate of MetBMI exhibited a significant decrease across all BMI classes (FIG. 5c), suggesting that metabolomics data captures information about the metabolic health response to the lifestyle intervention, beyond the baseline BMI class or the changes in BMI and other omic profiles.

Given the existence of multiple metabolic health sub-states within the standard BMI classes (FIG. 3), the difference between misclassification strata against the baseline MetBMI class was further investigated. In the normal baseline BMI class, while the mean estimate of the measured BMI remained constant in both Matched and Mismatched groups, the mean MetBMI estimate exhibited larger reduction in Mismatched group than Matched group (FIG. 5d), suggesting that the participants with MUNW phenotype improved their metabolic health to a greater extent than the participants with MHNW phenotype. Likewise, in the obese baseline BMI class, while the decrease in the mean estimate of the measured BMI was not significantly different between Matched and Mismatched groups at one year after the enrollment, the decrease in the mean MetBMI estimate was larger in Matched group than in Mismatched group (FIG. 5e), suggesting that the participants with MUO phenotype improved their metabolic health to a greater extent than the participants with MHO phenotype. Altogether, these results suggest that metabolic health was substantially improved during the program, in accordance with an individual's baseline metabolomic state, rather than with the individual's baseline BMI class.

### Example 19: Plasma Analyte Correlation Network in the Metabolically Obese Group Shifted Toward a Structure Observed in Metabolically Healthier State Following a Healthy Lifestyle Intervention

We explored longitudinal changes in plasma analyte correlation networks, focusing on the metabolically obese group. Based on the importance of the baseline metabolomic state (FIG. 5d, c), relationships between each plasma analyte-analyte correlation and the baseline MetBMI within the Arivale sub-cohort were assessed (FIG. 5a; 608 participants), using their interaction term in a generalized linear model (GLM; see above methods) of each analyte-analyte pair. In this type of model, the statistical test assesses whether the relationship between any two analytes is dependent on a third variable (in this case, the baseline MetBMI). Among 608,856 pairwise relationships of plasma analytes, 100 analyte-analyte correlation pairs, comprising 82 metabolites, 33 proteins, and 16 clinical laboratory tests, were significantly modified by the baseline MetBMI (FDR<0.05; Supplementary Data 7). Subsequently, longitudinal changes of these 100 pairs within the metabolically obese group were assessed (i.e., the baseline obese MetBMI class; 182 participants), using the interaction term (i.e., interaction with days in the program) in a generalized estimating equation (GEE; see above methods) of each analyte-analyte pair. Among the 100 pairs, 27 analyte-analyte correlation pairs were significantly modified by days in the program (FDR<0.05; FIG. 6a, Supplementary Data 7). These 27 pairs were mainly derived from metabolites (21 metabolites, 3 proteins, 3 clinical laboratory tests). One of these time-varying pairs was homoarginine and phenyllactate (PLA). Homoarginine was recently found to be a biomarker for CVD47 and was a robustly retained positive predictor in MetBMI and CombiBMI models (FIG. 2a and FIG. 11a). PLA is a gut microbiome-derived phenylalanine derivative known to have antimicrobial activity and antioxidant activity. The positive correlation between homoarginine and PLA was observed in the metabolically obese group at baseline (FIG. 6b) and became weaker in this group during the course of the intervention (FIG. 6c), implying that metabolic dysregulation specific to the metabolically obese group was somewhat improved during the program. Collectively, these findings indicate that metabolic improvement was not limited to changes in specific blood analyte concentrations but also changes in the association structure among analytes.

### Example 20: Discussion

Obesity is a significant risk factor for many chronic diseases. The heterogeneous nature of human health conditions, with variable manifestation ranging from metabolic abnormalities to cardiovascular symptoms, calls for deeper molecular characterizations in order to optimize wellness and reduce the current global epidemic of chronic diseases. In this study, it has been demonstrated that obesity profoundly perturbs human physiology, as reflected across all the studied omics modalities. The key findings of this study are: (1) machine learning-based multiomic BMI estimates were better suited to identifying heterogeneous metabolic health than the classically-measured BMI, while maintaining a high level of interpretability and intuitiveness attributed to the original metric (FIG. 1-3); (2) among all omics studied, metabolomic reflection of obesity exhibited the strongest correspondence to gut microbiome community structure (FIG. 4); (3) plasma metabolomics exhibited the strongest (and/or earliest) response to lifestyle coaching, while plasma proteomics exhibited a weaker (and/or more delayed) response than the measured BMI (FIG. 5b, c); (4) compared to the participants with metabolically healthy phenotype (i.e., BMI class=MetBMI class), the participants with metabolically unhealthy phenotype (i.e., BMI class<MetBMI class) exhibited a greater improvement in their metabolic health (but not in weight loss per se) in response to the healthy lifestyle coaching (FIG. 5d, c); (5) dozens of analyte-analyte associations were modified in the participants of the metabolically obese group (i.e., obese MetBMI class), following the healthy lifestyle intervention (FIG. 6).

Although BMI is used as a measure of obesity, fat distribution in the body is an important factor for understanding the heterogenous nature of obesity. In particular, abdominal obesity, which is characterized by excessive visceral fat (rather than subcutaneous fat) around the abdominal region, is known to be associated with chronic diseases such as MetS. Thus, abdominal obesity has been assessed by analyzing the anthropometric WHtR, which was highly correlated with BMI in the Arivale subcohort (Pearson's r=0.86; FIG. 13a-c). Omics-based WHtR models were generated (FIG. 13a, Supplementary Data 8), and obtained consistent findings to the omics-based BMI models (FIG. 13d-m). Interestingly, the majority of the retained analytes in each omics-based WHtR model was also retained in its corresponding omics-based BMI model with the similar feature importance (FIG. 14a-d). In addition, ΔWHtR was highly correlated with ABMI across all omics categories (FIG. 14c). Moreover, although the WC measurements were not available for the defined TwinsUK cohort, direct fat measurements of the android region by DXA were associated with MetBMI class in the TwinsUK cohort (FIG. 12c). Therefore, although BMI requires complementary information of the WC-related measurements for the diagnosis of abdominal obesity, the omics-based BMI model likely captures the obesity characteristics including abdominal obesity.

Multiple observational studies have explored obesity biomarkers. The involvements of insulin/insulin-like growth factor (IGF) axis and chronic low-grade inflammation have been discussed in the context of obesity-related disease risks, backed up by robust associations of obesity with IGFBP1/2 (−BMI), adipokines such as LEP (+BMI), adiponectin (−BMI), FABP4 (+BMI), and ADM (+BMI) and proinflammatory cytokines such as interleukin 6 (IL6; +BMI). Consistent with these well known associations, it was observed that positive BMI associations with LEP, FABP4, IL1RN, IL6, ADM, and insulin and negative BMI associations with IGFBP1/2 and adiponectin (FIG. 2c, d). Importantly, all these known biomarkers were incorporated into our omics-based BMI models, and most of them were consistently retained as important features of these models (FIG. 2a; FIG. 11b, c). At the same time, it was observed that RAGE explained a relatively small proportion of the variance in BMI (FIG. 2c), while being a strong negative predictive feature in all ten models of ProtBMI and CombiBMI (FIG. 2a, FIG. 11b). Soluble RAGE (sRAGE) has been gradually highlighted in the contexts of T2DM and CVD, with several reports on the negative association between sRAGE and BMI. Therefore, omics-inferred BMI may reflect not only obesity status but also the early transition towards clinical manifestations of obesity-related chronic diseases.

Likewise, many epidemiological studies have revealed metabolomic biomarkers for obesity. In line with these previous findings, it was confirmed positive BMI associations with mannose, uric acid (urate), and glutamate and negative BMI associations with asparagine and glycine (FIG. 2b). Furthermore, all of these metabolites were consistently incorporated into all ten models of MetBMI and CombiBMI (FIG. 2a, FIG. 11a). In addition, many lipids emerged as strong predictors in MetBMI and CombiBMI models; in particular, glycerophosphocholines (GPCs) were negative predictors in these models, while sphingomyelins (SMs) were positive predictors (FIG. 2a, FIG. 11a), even though both have a phosphocholine group in common. Although lipid has traditionally been regarded as a factor that is positively associated with obesity, recent metabolomics studies have revealed variable trends for different fatty acid species; e.g., plasma lysophosphatidylcholines (LPCs) are decreased in mice with obesity (high-fat diet model), which corresponded well with our results (e.g., LPC(18:1), described as 1-oleoyl-GPC(18:1) in FIG. 2b and FIG. 11b). However, because there are many combinations of acyl residues in lipids and many potential confounding factors with obesity, systematic understanding of the species-level lipid biomarkers for obesity remains challenging. Our approach, applying machine learning to metabolomics data, addresses this challenge by automatically and systematically providing a molecular signature of obesity, reflecting the versatile and complex metabolite species. Altogether, omics-based BMI models can be regarded as multidimensional profiles of obesity, possessing detailed mechanistic information.

Recently, Cirulli and colleagues have reported a machine learning model for estimating BMI, computed from blood metabolomics, which captured obesity-related phenotypes. Their main model explained 39.1% of the variance in BMI, while our MetBMI model explained 68.9% of the variance in BMI (FIG. 2b). Other than the difference in cohorts, the performance gap is likely a result of differences in modeling strategies. Cirulli and colleagues stringently selected 49 metabolites, out of their metabolomics panel of 1,007 metabolites, based on a pre-screening for significant adjusted associations with BMI, and subsequently applied a tenfold CV implementation of ridge or LASSO method. In contrast, the LASSO method was used for feature selection, applying it to our full metabolomics panel of 766 metabolites. In addition to the increased number of metabolites included in the model fitting, our higher performance may stem from the presence of metabolites which were critical for BMI prediction in a multivariate model, but not strongly associated with BMI on their own. Actually, similarly to the above example of RAGE in ProtBMI model, our MetBMI model contained multiple metabolites that were weakly associated with BMI but consistently retained across all ten models (FIG. 2b, FIG. 11a). At the same time, the majority of the 49 metabolites reported by Cirulli and colleagues (14-20 metabolites among the 31-41 corresponding metabolites in our metabolomics panel) were retained in at least one of the ten MetBMI models. Therefore, our strategy of feature selection through machine learning, without a pre-filtering strep, may be preferable for predicting BMI from metabolomics.

A recent study investigating multiomic changes in response to weight perturbations demonstrated that some weight gain-associated blood signatures were reversed during subsequent weight loss, while others persisted. Interestingly, MetBMI was more responsive to the healthy lifestyle intervention than the measured BMI or ChemBMI, while ProtBMI was more resistant to the same intervention (FIG. 5b, c). Our analyses of the predictors in the omics-based BMI models (FIG. 2; FIG. 8c-h, FIG. 11) suggested that the distribution of feature importance among metabolites was considerably wider, while only a small subset of measured proteins (˜5 proteins) was predominantly reflective of obesity profiles. Therefore, the effect of lifestyle coaching may consist of small additive contributions in blood metabolites in the short term. However, a longer longitudinal analysis is needed to infer the physiological meaning of these omics-dependent dynamics. For instance, it is possible that ProtBMI shows a delayed response to weight loss (over a span greater than a year measured presently; FIG. 5b, c), indicating blood metabolites and proteins may be early and late responders to a lifestyle intervention, respectively, such as in the case of the changes in blood glucose compared to the changes in HbA1c when assessing glucose homeostasis. If the difference between the measured and omics-inferred BMIs remains constant even after one year, blood metabolites and proteins would appear to be more and less sensitive to weight loss than the measured BMI, respectively. In either scenario, monitoring blood multiomics during weight loss programs could help participants maintain their motivation to stay engaged with persistent lifestyle changes, because they would receive rapid feedback on how lifestyle changes were impacting their health, even in the absence of weight loss. In addition, long-term maintenance of the improvement is an important challenge for lifestyle interventions; although there is variability between prior reports, one study estimated that only ˜20% of the individuals with overweight successfully maintain their weight loss in post-intervention. Despite this relatively low rate of long-term success, there is evidence that lifestyle interventions had benefits in preventing diabetes incidence as far as 20 years post intervention, even if weight was regained. The observed larger improvement of MetBMI compared to the measured BMI could potentially contribute to this protective long-term effect, persisting even when weight is regained. Further investigation is required, especially with regard to the long-term dynamics of MetBMI and ProtBMI responses, which may provide a foothold in developing scientific strategies aimed at long-term maintenance of metabolic health.

### REFERENCES

1. NCD Risk Factor Collaboration (NCD-RisC). Trends in adult body-mass index in 200 countries from 1975 to 2014: a pooled analysis of 1698 population-based measurement studies with 19.2 million participants. Lancet (London, England) 387, 1377-1396 (2016).
2. NCD Risk Factor Collaboration (NCD-RisC). Worldwide trends in body-mass index, underweight, overweight, and obesity from 1975 to 2016: a pooled analysis of 2416 population-based measurement studies in 128.9 million children, adolescents, and adults. Lancet (London, England) 390, 2627-2642 (2017).
3. Kopelman, P. G. Obesity as a medical problem. Nature 404, 635-43 (2000).
4. Haslam, D. W. & James, W. P. T. Obesity. Lancet (London, England) 366, 1197-209 (2005).
5. Kahn, S. E., Hull, R. L. & Utzschneider, K. M. Mechanisms linking obesity to insulin resistance and type 2 diabetes. Nature 444, 840-6 (2006).
6. Van Gaal, L. F., Mertens, I. L. & De Block, C. E. Mechanisms linking obesity with cardiovascular disease. Nature 444, 875-80 (2006).
7. Magkos, F. et al. Effects of Moderate and Subsequent Progressive Weight Loss on Metabolic Function and Adipose Tissue Biology in Humans with Obesity. Cell Metab. 23, 591-601 (2016).
8. Hamman, R. F. et al. Effect of weight loss with lifestyle intervention on risk of diabetes. Diabetes Care 29, 2102-7 (2006).
9. Sun, Q. et al. Comparison of dual-energy x-ray absorptiometric and anthropometric measures of adiposity in relation to adiposity-related biologic factors. Am. J. Epidemiol. 172, 1442-54 (2010).
10. Prentice, A. M. & Jebb, S. A. Beyond body mass index. Obes. Rev. 2, 141-7 (2001).
11. Okorodudu, D. O. et al. Diagnostic performance of body mass index to identify obesity as defined by body adiposity: A systematic review and meta-analysis. Int. J. Obes. 34, 791-799 (2010).
12. WHO Expert Consultation. Appropriate body-mass index for Asian populations and its implications for policy and intervention strategies. Lancet (London, England) 363, 157-63 (2004).
13. Ruderman, N., Chisholm, D., Pi-Sunyer, X. & Schneider, S. The metabolically obese, normal-weight individual revisited. Diabetes 47, 699-713 (1998).
14. Ding, C., Chan, Z. & Magkos, F. Lean, but not healthy: the ‘metabolically obese, normal-weight’ phenotype. Curr. Opin. Clin. Nutr. Metab. Care 19, 408-417 (2016).
15. Smith, G. I., Mittendorfer, B. & Klein, S. Metabolically healthy obesity: facts and fantasies. J. Clin. Invest. 129, 3978-3989 (2019).
16. Appleton, S. L. et al. Diabetes and cardiovascular disease outcomes in the metabolically healthy obese phenotype: a cohort study. Diabetes Care 36, 2388-94 (2013).
17. Schröder, H. et al. Determinants of the transition from a cardiometabolic normal to abnormal overweight/obese phenotype in a Spanish population. Eur. J. Nutr. 53, 1345-53 (2014).
18. Williams, S. A. et al. Plasma protein patterns as comprehensive indicators of health. Nat. Med. 25, 1851-1857 (2019).
19. Bar, N. et al. A reference map of potential determinants for the human serum metabolome. Nature 588, 135-140 (2020).
20. Wilmanski. T. et al. Blood metabolome predicts gut microbiome α-diversity in humans. Nat. Biotechnol. 37, 1217-1228 (2019).
21. Cirulli, E. T. et al. Profound Perturbation of the Metabolome in Obesity Is Associated with Health Risk. Cell Metab. 29, 488-500.e2 (2019).
22. Talmor-Barkan, Y. et al. Metabolomic and microbiome profiling reveals personalized risk factors for coronary artery disease. Nat. Med. 28, 295-302 (2022).
23. Nimptsch, K., Konigorski, S. & Pischon, T. Diagnosis of obesity and use of obesity biomarkers in science and clinical medicine. Metabolism. 92, 61-70 (2019).
24. Price, N. D. et al. A wellness study of 108 individuals using personal, dense, dynamic data clouds. Nat. Biotechnol. 35, 747-756 (2017).
25. Zubair, N. et al. Genetic Predisposition Impacts Clinical Changes in a Lifestyle Coaching Program. Sci. Rep. 9, 6805 (2019).
26. Earls, J. C. et al. Multi-Omic Biological Age Estimation and Its Correlation With Wellness and Disease Phenotypes: A Longitudinal Study of 3,558 Individuals. J. Gerontol. A. Biol. Sci. Med. Sci. 74, S52-S60 (2019).
27. Wainberg, M. et al. Multiomic blood correlates of genetic risk identify presymptomatic disease alterations. Proc. Natl. Acad. Sci. U.S.A 117, 21813-21820 (2020).
28. Wilmanski, T. et al. Gut microbiome pattern reflects healthy ageing and predicts survival in humans. Nat. Metab. 3, 274-286 (2021).
29. Zimmer, A. et al. The geometry of clinical labs and wellness states from deeply phenotyped humans. Nat. Commun. 12, 3578 (2021).
30. Tibshirani, R. Regression Shrinkage and Selection Via the Lasso. J. R. Stat. Soc. Ser. B 58, 267-288 (1996).
31. Moayyeri, A., Hammond, C. J., Valdes, A. M. & Spector, T. D. Cohort Profile: TwinsUK and healthy ageing twin study. Int. J. Epidemiol. 42, 76-85 (2013).
32. Long. T. et al. Whole-genome sequencing identifies common-to-rare variants associated with human blood metabolites. Nat. Genet. 49, 568-578 (2017).
33. Xu. X. et al. Habitual sleep duration and sleep duration variation are independently associated with body mass index. Int. J. Obes. (Lond). 42, 794-800 (2018).
34. Stefan, N., Schick, F. & Häring, H.-U. Causes, Characteristics, and Consequences of Metabolically Unhealthy Normal Weight in Humans. Cell Metab. 26, 292-300 (2017).
35. Blüher, M. Metabolically Healthy Obesity. Endocr. Rev. 41, 405-420 (2020).
36. Shah, N. R. & Braverman, E. R. Measuring adiposity in patients: the utility of body mass index (BMI), percent body fat, and leptin. PLOS One 7, e33308 (2012).
37. Tomiyama, A. J., Hunger, J. M., Nguyen-Cuu, J. & Wells, C.

Misclassification of cardiometabolic health when using body mass index categories in NHANES 2005-2012. Int. J. Obes. (Lond). 40, 883-6 (2016).
38. Bennett, C. M., Guo, M. & Dharmage, S. C. HbA(1c) as a screening tool for detection of Type 2 diabetes: a systematic review. Diabet. Med. 24, 333-43 (2007).
39. Pereira-Santos, M., Costa, P. R. F., Assis, A. M. O., Santos, C. A. S. T. & Santos, D. B. Obesity and vitamin D deficiency: a systematic review and meta-analysis. Obes. Rev. 16, 341-9 (2015).
40. Ridaura, V. K. et al. Gut microbiota from twins discordant for obesity modulate metabolism in mice. Science 341, 1241214 (2013).
41. Turnbaugh, P. J. et al. A core gut microbiome in obese and lean twins. Nature 457, 480-484 (2009).
42. Le Chatelier, E. et al. Richness of human gut microbiome correlates with metabolic markers. Nature 500, 541-546 (2013).
43. Walters, W. A., Xu, Z. & Knight, R. Meta-analyses of human gut microbes associated with obesity and IBD. FEBS Lett. 588, 4223-4233 (2014).
44. Duvallet, C., Gibbons, S. M., Gurry, T., Irizarry, R. A. & Alm, E. J. Meta-analysis of gut microbiome studies identifies disease-specific and shared responses. Nat. Commun. 8, 1784 (2017).
45. Visconti, A. et al. Interplay between the human gut microbiome and host metabolism. Nat. Commun. 10, 4505 (2019).
46. Diener, C. et al. Baseline Gut Metagenomic Functional Gene Signature Associated with Variable Weight Loss Responses following a Healthy Lifestyle Intervention in Humans. mSystems 6, e0096421 (2021).
47. Karetnikova, E. S. et al. Is Homoarginine a Protective Cardiovascular Risk Factor? Arterioscler. Thromb. Vasc. Biol. 39, 869-875 (2019).
48. Dieuleveux, V., Lemarinier, S. & Guéguen, M. Antimicrobial spectrum and target site of D-3-phenyllactic acid. Int. J. Food Microbiol. 40, 177-83 (1998).
49. Beloborodova, N. et al. Effect of phenolic acids of microbial origin on production of reactive oxygen species in mitochondria and neutrophils. J. Biomed. Sci. 19, 89 (2012).
50. Després, J.-P. & Lemieux, I. Abdominal obesity and metabolic syndrome. Nature 444, 881-7 (2006).
51. Ashwell, M., Gunn, P. & Gibson, S. Waist-to-height ratio is a better screening tool than waist circumference and BMI for adult cardiometabolic risk factors: systematic review and meta-analysis. Obes. Rev. 13, 275-86 (2012).
52. Swainson, M. G., Batterham, A. M., Tsakirides, C., Rutherford, Z. H. & Hind, K. Prediction of whole-body fat percentage and visceral adipose tissue mass from five anthropometric variables. PLOS One 12, e0177175 (2017).
53. Li. Y. et al. Adrenomedullin is a novel adipokine: adrenomedullin in adipocytes and adipose tissues. Peptides 28, 1129-43 (2007).
54. Egaña-Gorroño, L. et al. Receptor for Advanced Glycation End Products (RAGE) and Mechanisms and Therapeutic Opportunities in Diabetes and Cardiovascular Disease: Insights From Human Subjects and Animal Models. Front. Cardiovasc. Med. 7, 37 (2020).
55. Norata, G. D. et al. Circulating soluble receptor for advanced glycation end products is inversely associated with body mass index and waist/hip ratio in the general population. Nutr. Metab. Cardiovasc. Dis. 19, 129-34 (2009).
56. Rauschert, S., Uhl, O., Koletzko, B. & Hellmuth, C. Metabolomic biomarkers for obesity in humans: A short review. Ann. Nutr. Metab. 64, 314-324 (2014).
57. Rangel-Huerta, O. D., Pastor-Villaescusa, B. & Gil, A. Are we close to defining a metabolomic signature of human obesity? A systematic review of metabolomics studies. Metabolomics 15, 93 (2019).
58. Barber, M. N. et al. Plasma lysophosphatidylcholine levels are reduced in obesity and type 2 diabetes. PLOS One 7, e41456 (2012).
59. Piening. B. D. et al. Integrative Personal Omics Profiles during Periods of Weight Gain and Loss. Cell Syst. 6, 157-170.e8 (2018).
60. Koenig. R. J. et al. Correlation of glucose regulation and hemoglobin Alc in diabetes mellitus. N. Engl. J. Med. 295, 417-20 (1976).
61. Wing. R. R. & Phelan, S. Long-term weight loss maintenance. Am. J. Clin. Nutr. 82, 222S-225S (2005).
62. Li, G. et al. The long-term effect of lifestyle interventions to prevent diabetes in the China Da Qing Diabetes Prevention Study: a 20-year follow-up study. Lancet (London, England) 371, 1783-9 (2008).
63. Diabetes Prevention Program Research Group et al. 10-year follow-up of diabetes incidence and weight loss in the Diabetes Prevention Program Outcomes Study. Lancet (London, England) 374, 1677-86 (2009).
64. Yilmaz, P. et al. The SILVA and ‘All-species Living Tree Project (LTP)’ taxonomic frameworks. Nucleic Acids Res. 42, D643-8 (2014).
65. Chen, S., Zhou, Y., Chen, Y. & Gu, J. fastp: an ultra-fast all-in-one FASTQ preprocessor. Bioinformatics 34, 1884-1890 (2018).
66. Lu, J. et al. Metagenome analysis using the Kraken software suite. Nat. Protoc. 1-25 (2022). doi: 10.1038/s41596-022-00738-y
67. Stekhoven, D. J. & Bühlmann, P. Missforest-Non-parametric missing value imputation for mixed-type data. Bioinformatics 28, 112-118 (2012).
68. Robin, X. et al. pROC: an open-source package for R and S+ to analyze and compare ROC curves. BMC Bioinformatics 12, 77 (2011).
69. DeLong, E. R., DeLong, D. M. & Clarke-Pearson, D. L. Comparing the areas under two or more correlated receiver operating characteristic curves: a nonparametric approach. Biometrics 44, 837-45 (1988).
70. Gu, Z., Gu, L., Eils, R., Schlesner, M. & Brors, B. Circlize implements and enhances circular visualization in R. Bioinformatics 30, 2811-2812 (2014).

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

What is claimed is:

1. A computer-implemented method of determining an omics-inferred anthropomorphic body index of a subject, the computer comprising one or more processors programmed to perform a series of steps, comprising:
(a) accessing blood analyte omics data of the subject;
(b) generating an omics body index for the subject by applying a machine learning model to the subject omics data, the machine learning model fitted to blood analyte omic and anthropomorphic body index data of a reference population, the reference population comprising a heterogeneous mixture of individuals classified by different anthropomorphic body index classes;
(c) classifying the subject by the omics body index class according to the anthropomorphic body index class boundaries; and
(d) outputting the omics body index class for the subject.

2. The method of claim 1, wherein the anthropomorphic body index is selected from body mass index (BMI, kg m-2), waist circumference (cm), and waist-to-height ratio (WHtR, unitless).

3. The method of claim 2, wherein the anthropomorphic BMI is a World Health Organization (WHO) standard having class boundaries selected from: underweight <18.5 kg m-2; normal 18.5 to 25 kg m-2; overweight 25 to 30 kg m-2; and obese ≥30 kg m-2.

4. The method of claim 3, wherein the WHO anthropomorphic BMI standard further comprises class boundaries selected from: severely underweight <16.5 kg/m\hat{ }2; class 1 obesity 30 to <35 kg m-2; class 2 obesity 35 to <40 kg m-2; and class 3 obesity 40 kg m-2 or higher.

5. The method of claim 2, wherein the anthropomorphic BMI is an Asian-Pacific standard having class boundaries selected from: underweight <18.5 kg m-2; normal 18.5 to 22.9 kg m-2; overweight 23 to 24.9 kg m-2; and obese ≥25 kg m-2.

6. The method of claim 2, wherein the WHtR is a United Kingdom National Institute for Health and Care Excellence (NICE) standard having class boundaries selected from: 0.4 to 0.49 WHtR for healthy central adiposity; 0.5 to 0.59 WHtR for increased central adiposity; and, 0.6 or more WHtR for high central adiposity.

7. The method of claim 1, the method further comprising:
outputting feedback on the omics body index class selected from, or comprising: (i) health intervention potential, (ii) recommended health intervention, and (iii) feedback on efficacy of the health intervention potential and/or the recommended health intervention.

8. The method of claim 7, wherein (i) the health intervention potential is weight loss potential and/or omic body index reduction potential, (ii) the recommended health intervention is a lifestyle intervention, and (iii) the feedback on efficacy comprises a comparison of the subject omics body index before, after, or before and after the health intervention.

9. The method of claim 7, wherein the feedback is a longitudinal trajectory.

10. The method of claim 7, wherein the recommended health intervention is a lifestyle change, such as regular exercise, prebiotics, probiotics, supplements, and prescribed medical treatment compliance.

11. The method of claim 1, wherein the blood analyte omics data of the reference population comprises a panel of ten or more analytes selected from, or comprising, metabolomic data, proteomic data, or a combination thereof.

12. The method of claim 11, wherein step (a) further comprises accessing clinical labs data of the subject, and wherein step (b) further comprises generating an omic body index for the subject by applying the machine learning model to the omics and clinical labs data of the subject, the machine learning model fitted to the blood analyte omic and clinical labs data of the reference population.

13. The method of claim 12, wherein the machine learning model is fitted to omics data comprising, or selected from, metabolomic data (MetBMI model, or MetWHtR in case of WHtR), and proteomic data (ProBMI model), clinical labs data (ChemBMI model), or a combination thereof (CombiBMI model).

14. The method of claim 11, wherein the blood analyte omics data of the subject comprises the metabolomic data and/or analytes co-linear therewith.

15. The method of claim 1, wherein the blood analyte omic data of the reference population or the subject comprises actual and imputed data, such as imputation by random forest regression or k-nearest neighbors (kNN).

16. A system comprising:
one or more data processors; and
a non-transitory computer readable storage medium containing instructions which, when executed on the one or more data processors, cause the one or more data processors to perform a set of actions including:
(a) accessing blood analyte omics data of the subject;
(b) generating an omics body index for the subject by applying a machine learning model to the subject omics data, the machine learning model fitted to blood analyte omic and anthropomorphic body index data of a reference population, the reference population comprising a heterogeneous mixture of individuals classified by different anthropomorphic body index classes;
(c) classifying the subject by the omics body index class according to the anthropomorphic body index class boundaries; and
(d) outputting the omics body index class for the subject.

21. A computer-program product tangibly embodied in a non-transitory machine-readable storage medium, including instructions configured to cause one or more data processors to perform a set of actions including:
(a) accessing blood analyte omics data of the subject;
(b) generating an omics body index for the subject by applying a machine learning model to the subject omics data, the machine learning model fitted to blood analyte omic and anthropomorphic body index data of a reference population, the reference population comprising a heterogeneous mixture of individuals classified by different anthropomorphic body index classes;
(c) classifying the subject by the omics body index class according to the anthropomorphic body index class boundaries; and
(d) outputting the omics body index class for the subject.
